SEQUENCE LISTING (24 sequences)

SEQ ID NO: 1 - protein, 9 aa, Homo sapiens
  MOD_RES (9)..(9): AMIDATION
CYIQNCPLG

SEQ ID NO: 2 - protein, 10 aa, Artificial sequence
  Oxytocin fragments and variants
  MISC_FEATURE (1)..(1): X1 can be Cys or nothing
  MISC_FEATURE (2)..(2): X2 can be Tyr, Phe or nothing
  MISC_FEATURE (3)..(3): X3 can be Ile, Val, Homophenylalanine, Phe, Cyclohexylalanine, or nothing
  MISC_FEATURE (4)..(4): X4 can be Gln, Ser, Thr, Citruline, Arg or Diaminobutyric acid
  MISC_FEATURE (7)..(7): X5 (position 7) can be Pro or nothing
  MISC_FEATURE (8)..(8): X6 (position 8) can be Ile, Leu, nothing, Val, Homoserine, Diaminobutyric acid, Thr, Arg or Citruline
  MISC_FEATURE (9)..(9): X7 (position 9) can be Gly, nothing or Ala
  MOD_RES (10)..(10): AMIDATION
  MISC_FEATURE (10)..(10): X8 (position 10) can be Gly or nothing
XXXXNCXXXX

SEQ ID NO: 3 - protein, 9 aa, UNKNOWN
  Present in most bird species
  MOD_RES (9)..(9): AMIDATION
CYIQNCPIG

SEQ ID NO: 4 - protein, 9 aa, UNKNOWN
  Present in most bony fish species
  MOD_RES (9)..(9): AMIDATION
CYISNCPIG

SEQ ID NO: 5 - protein, 9 aa, Eisenia foetida
  MOD_RES (9)..(9): AMIDATION
CFVRNCPTG

SEQ ID NO: 6 - protein, 9 aa, UNKNOWN
  Present in most bird species
  MOD_RES (9)..(9): AMIDATION
CYIQNCPRG

SEQ ID NO: 7 - protein, 9 aa, Homo sapiens
  MOD_RES (9)..(9): AMIDATION
CYFQNCPRG

SEQ ID NO: 8 - protein, 6 aa, Artificial sequence
  Oxytocin fragment
  MOD_RES (6)..(6): AMIDATION
CYIQNC

SEQ ID NO: 9 - protein, 7 aa, Artificial sequence
  oxytocin fragment
  MOD_RES (7)..(7): AMIDATION
CYIQNCP

SEQ ID NO: 10 - protein, 8 aa, Artificial sequence
  Oxytocin fragment
  MOD_RES (8)..(8): AMIDATION
CYIQNCPL

SEQ ID NO: 11 - protein, 8 aa, Artificial sequence
  Oxytocin fragment
  MOD_RES (8)..(8): AMIDATION
YIQNCPLG

SEQ ID NO: 12 - protein, 7 aa, Artificial sequence
  Oxytocin fragment
  MOD_RES (7)..(7): AMIDATION
IQNCPLG

SEQ ID NO: 13 - protein, 6 aa, Artificial sequence
  Oxytocin fragment
  MOD_RES (6)..(6): AMIDATION
QNCPLG

SEQ ID NO: 14 - protein, 5 aa, Artificial sequence
  Oxytocin fragment
  MOD_RES (5)..(5): AMIDATION
IQNCP

SEQ ID NO: 15 - protein, 10 aa, Artificial sequence
  Oxytocin variant
  MOD_RES (10)..(10): AMIDATION
CYIQNCPLGG

SEQ ID NO: 16 - protein, 6 aa, Artificial sequence
  Oxytocin variant
  MOD_RES (6)..(6): AMIDATION
QNCPLL

SEQ ID NO: 17 - protein, 9 aa, Artificial sequence
  Oxytocin variant
  MOD_RES (9)..(9): AMIDATION
CYVTNCPLG

SEQ ID NO: 18 - protein, 9 aa, Artificial sequence
  Oxytocin variant
  MISC_FEATURE (3)..(3): X is Homophenylalanine
  MOD_RES (9)..(9): AMIDATION
CYXTNCPVG

SEQ ID NO: 19 - protein, 9 aa, Artificial sequence
  Oxytocin variant
  MISC_FEATURE (4)..(4): X is Citruline
  MOD_RES (9)..(9): AMIDATION
CYFXNCPLG

SEQ ID NO: 20 - protein, 9 aa, Artificial sequence
  Oxytocin variant
  MISC_FEATURE (3)..(3): X is Cyclohexylalanine
  MISC_FEATURE (8)..(8): X is Homoserine
  MOD_RES (9)..(9): AMIDATION
CYXRNCPXA

SEQ ID NO: 21 - protein, 9 aa, Artificial sequence
  Oxytocin variant
  MISC_FEATURE (4)..(4): X is Diaminobutyric acid
  MISC_FEATURE (8)..(8): X is Diaminobutyric acid
  MOD_RES (9)..(9): AMIDATION
CYVXNCPXA

SEQ ID NO: 22 - protein, 9 aa, Artificial sequence
  Oxytocin variant
  MISC_FEATURE (3)..(3): X is Homophenylalanine
  MISC_FEATURE (4)..(4): X is Diaminobutyric acid
  MISC_FEATURE (8)..(8): X is Citruline
  MOD_RES (9)..(9): AMIDATION
CYXXNCPXA

SEQ ID NO: 23 - protein, 9 aa, Artificial sequence
  Oxytocin variant
  MOD_RES (9)..(9): AMIDATION
CYFRNCPVA

SEQ ID NO: 24 - protein, 9 aa, Artificial sequence
  Oxytocin variant
  MISC_FEATURE (3)..(3): X is Cyclohexylalanine
  MISC_FEATURE (4)..(4): X is Citruline
  MOD_RES (9)..(9): AMIDATION
CYXXNCPRG

(19) United States
(12) Patent Application Publication
(10) Pub. No.: US 2017/0232059 A1
(43) Pub. Date: Aug. 17, 2017

(54) COMPOSITION COMPRISING OXYTOCIN

(71) Applicant: PEP-TONIC MEDICAL AB, Vaxjo (SE)

(72) Inventor: Kerstin Uvnäs-Moberg, Djursholm (SE)

(21) Appl. No.: 14/435,196

(22) PCT Filed: Oct. 11, 2013
(86) PCT No.: PCT/EP2013/017291

§ 371 Date: Oct. 4, 2016

Related U.S. Application Data
(60) Provisional application No. 61/712,876, filed on Oct. 12, 2012.
(60) Provisional application No. 61/712,867, filed on Oct. 12, 2012.

(51) Int. Cl. A61K 38/11; A61K 47/38; A61K 9/00

(52) U.S. Cl. CPC A61K 38/11; A61K 9/0014; A61K 9/0034; A61K 47/38

(57) ABSTRACT

The present document relates to the field of pharmaceutical composition comprising oxytocin and/or one or more fragment(s) and/or variant(s) thereof, for use in the treatment and/or prevention of a disorder which is responsive to modulation of the endogenous oxytocin production.

### TECHNICAL FIELD

The present document relates to the field of pharmaceuticals. More specifically the present document relates to new uses of pharmaceutical compositions comprising oxytocin and/or one or more fragment(s) and/or variant(s) thereof.

### BACKGROUND

Oxytocin was one of the first peptide hormones to be isolated and sequenced. It is a nonapeptide with two cysteine residues that form a disulfide bridge between positions 1 and 6 and corresponds to the formula

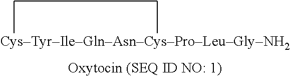

For a long time the only effects attributed to oxytocin were its stimulating effects on milk ejection and uterine contractions, but in the past decades it has been shown that oxytocin exerts a wide spectrum of effects within the central nervous system, CNS. It has been suggested that oxytocin participates in the control of memory and learning processes and of various types of behaviour such as feeding, locomotion, as well as maternal and sexual behaviour. Oxytocin is also suggested to participate in the control of cardiovascular functions, thermoregulation, and pain threshold and fluid balance. There is also evidence that oxytocin is involved in the control of various immunological processes. These findings have been confirmed in studies by administration of oxytocin to male subjects by the use of an oxytocin containing nasal spray. It has also been demonstrated that oxytocin injections cause a lowering of blood pressure and increased weight gain with long lasting effects after repetitive administration. As a central stimulating substance, oxytocin plays an important role in the interaction between mother and progeny in mammals. Oxytocin may also be used as prophylactics in young human beings e.g. already in new born babies or young children to prevent the development of diseases later on in life, which diseases are dependent on stress conditions during the fetal life. Such conditions may be heart/vessel diseases such as stroke, heart infarction, hypertension, and diabetes type 2.

In the human body oxytocin is produced in the paraventricular nucleus, PVN, and the supraoptic nucleus, SON, of the hypothalamus. It differs by only two amino acids from vasopressin, which is also produced in these nuclei. The magnocellular oxytocinergic neurones of the SON and PVN send axons to the posterior pituitary from which oxytocin is released into the circulation. Parvocellular oxytocinergic neurones that originate in the PVN project into multiple areas within CNS. The oxytocin-producing cells are innervated by cholinergic, catecholaminergic, serotonergic, dopaminergic as well as peptidergic neurones. The presence of oxytocin in different tissues outside the brain, such as the uterus, ovaries, testis, thymus, adrenal medulla and pancreas has been demonstrated and oxytocin is suggested to be produced in and to exert local effects in these organs. A parallel secretion of oxytocin into the brain regions and into the circulation occurs in response to some stimuli such as suckling, but other stimuli can cause separate activation of oxytocinergic neurones, terminating in the brain or the pituitary.

Previously, oxytocin has been shown to have a positive effect on many medical conditions, such as wound healing, cancer in situ and cervicitis, inflammation etc., in the context of a local effect. Further, the substance carbetocin has also been mentioned as a therapeutic as an oxytocin analogue. However, it is not appropriate to compare oxytocin and carbetocin when it comes to pharmacokinetics and the ability to cross natural membranes, as the half-life of oxytocin is about 30 min, while the half-life of carbetocin is about three times longer in addition to several structural differences. Therefore, the use of oxytocin is a challenge.

Further, Jonas et al. (Breastfeeding medicine, 2009, Vol. 4, No: 2) described the effects of intrapartum oxytocin administration and epidural analgesia on the concentration of plasma oxytocin and prolactin in response to suckling during the second day postpartum. Therein, oxytocin was administered intravenously and intramuscularly to investigate the effect on endogenous production of oxytocin in a dose-dependent manner. However, the intravenous administration of oxytocin was shown to provide an inhibiting effect on the endogenous oxytocin production. Accordingly, intravenously administrated oxytocin into the blood circulation in the doses of Jonas et al. can cause negative effects on the endogenous production of oxytocin.

Hence, there is still a need within the art to provide new treatments comprising the administration of oxytocin.

### SUMMARY OF INVENTION

It is thus an object of the present document to provide a pharmaceutical composition comprising oxytocin and/or one or more fragment(s) and/or variant(s) thereof, for use in the treatment and/or prevention of a disorder which is responsive to modulation of the endogenous oxytocin production. The treatment and/or prevention may further comprise the healing of a wound, and/or treating and/or preventing an atrophic condition in the skin or mucosa, such as treating and/or preventing vaginal atrophy. The composition may be administered topically, such as epicutaneously, sublingually, buccally, vaginally and/or anally. The composition may be administered once daily.

The present document is also directed to provide a method for the treatment and/or prevention of a disorder which is responsive to modulation of the endogenous oxytocin production in a subject comprising administrating a therapeutically effective amount of a composition comprising oxytocin and/or one or more fragment(s) and/or variant(s) thereof to said subject in need thereof. The composition in such a method may be administered topically, such as epicutaneously, sublingually, buccally, vaginally and/or anally. The composition may be administered once daily. Such a method may further comprise the healing of a wound, and/or treating and/or preventing an atrophic condition in the skin or mucosa, such as treating and/or preventing vaginal atrophy.

The present document is also directed to the use of oxytocin and/or one or more fragment(s) and/or variant(s) thereof, in the manufacture of a composition for the treatment and/or prevention of a disorder which is responsive to modulation of the endogenous oxytocin production. Such a use may further comprise the healing of a wound, and/or treating and/or preventing an atrophic condition in the skin or mucosa, such as treating and/or preventing vaginal atrophy. The composition may be administered topically, such as epicutaneously, sublingually, buccally, vaginally and/or anally. The composition may be administered once daily.

The disorder may be selected from the group consisting of: oxytocin deficiency, low social ability, anxiety, low energy level, stress sensitivity, mental climacteric symptoms, obsessive-compulsive disorder (OCD), borderline, attachment disorders, depression, autism and other forms of mental fragility or illness, pain, inflammation, an abusive disorder and/or disturbances of cardiovascular and gastrointestinal function of central origin, in particular an oxytocin deficiency.

The present document is also directed to a kit of parts comprising: a composition comprising oxytocin and/or one or more fragment(s) and/or variant(s) thereof, a container for said composition, and optionally instructions for use and/or means for applying said composition to a subject.

The oxytocin and/or one or more fragment(s) and/or variant(s) thereof in the composition may be administered in a dose of about 10-10 000 IU. Exemplary doses are about 100-6 000 IU, about 1 000-6 000 IU or about 2 000-4 000 IU, such a dose being particularly suitable for epicutaneous, sublingual or buccal administration. Other exemplary doses are about 10-600 IU, about 100-600 IU or about 200-400 IU, such doses being particularly suitable for vaginal or anal administration.

The composition may be a topical composition, such as an epicutaneous, sublingual, buccal, vaginal, or anal composition. The composition may be administered once a day.

The one or more fragment(s) and/or variant(s) of oxytocin may correspond to;

(SEQ ID NO: 2)
X₁-X₂-X₃-X₄-Asn-Cys-X₅-X₆-X₇-X₈-NH₂

wherein X₁ is selected from the group consisting of Cys and nothing;
X₂ is selected from the group consisting of Tyr, Phe, and nothing;
X₃ is selected from the group consisting of Ile, Val, Hoph, Phe, Cha, and nothing;
X₄ is selected from the group consisting of Gln, Ser, Thr, Cit, Arg, and Daba;
X₅ is selected from the group consisting of Pro and nothing;
X₆ is selected from the group consisting of Ile, Leu, nothing, Val, Hos, Daba, Thr, Arg, and Cit;
X₇ is selected from the group consisting of Gly, nothing, and Ala;
X₈ is selected from the group consisting of Gly and nothing.

The one or more fragment(s) and/or variant(s) of oxytocin is/are selected from the group consisting of the peptides corresponding to SEQ ID NO:3-24.

The composition may have a pH from about 3 to about 5, such as from about 3 to about 4.

The composition may further comprise an epithelium penetration enhancer. The epithelium penetration enhancer may e.g. be propylene glycol (PG), SDS, isopropyl myristate, ethanol, diethylene glycol ethyl ether and/or cineole.

The composition may further comprise at least one non-ionic cellulose ether, such as a non-ionic cellulosic ether selected from the group consisting of methyl cellulose (MC), hydroxypropylmethylcellulose (HPMC), hydroxypropylcellulose (HPC), hydroxypropylmethylcellulose (HPMC), hydroxyethylethylcellulose (HEEC) and hydroxyethylmethyl cellulose (HEMC), in particular hydroxypropylmethylcellulose (HPMC).

The composition may e.g. comprise oxytocin (SEQ ID NO:1) and hydroxypropylmethylcellulose (HPMC).

An exemplary composition comprises:

i) about 0.1 to 2 mg/ml, 0.17-1 mg/ml, 0.17-0.68 mg/ml, or 0.34-0.68 mg/ml oxytocin and/or one or more fragment(s) and/or variant(s) of oxytocin;
ii) a non-ionic cellulose ether, such as hydroxypropylmethylcellulose (HPMC); said composition having a pH of about 3-5, such as about 3-4.

Another exemplary composition comprises:

i) about 1 to 15 mg/ml, 1.7-10 mg/ml, 1.7-6.8 mg/ml, or 3.4-6.8 mg/ml oxytocin;
ii) a non-ionic cellulose ether, such as hydroxypropylmethylcellulose (HPMC);

said composition having a pH of about 3-5, such as about 3-4.

The composition may further comprise an epithelium penetration enhancer, such as propylene glycol (PG), SDS, isopropyl myristate, ethanol, diethylene glycol ethyl ether and/or cineole.

### Definitions

Whenever “oxytocin”, “oxytocin peptide” and/or “oxytocin molecule” is referred to herein, this encompasses oxytocin (SEQ ID NO:1) and/or one or more fragment(s) and/or variant(s) thereof as defined herein according to the general formula of SEQ ID NO:2, or any other variant and/or fragment as mentioned herein, as well as homologues thereof, as well as pharmaceutically acceptable salts thereof. Whenever a fragment, variant or homologue of an oxytocin molecule/peptide is envisaged it is to be understood that such a variant, fragment or homologue encompasses a biological activity comparable to the oxytocin molecule itself (SEQ ID NO:1). As an example, it can be shown that a substance has oxytocin activity by performing tests showing the activity of the actual substance, e.g. by performing a double blind cross-over randomised protocol as described in Example 1 of WO0178758.

Accordingly, a “variant” of oxytocin as referred to herein, refers to a peptide which has been varied in its amino acid structure as compared to the oxytocin molecule in that some amino acid positions may have been altered by introducing other amino acids in such positions, such as natural or unnatural amino acids as exemplified herein, or it may have been extended by adding one or more natural or unnatural amino acid(s) to either ends of the peptide. In addition, other structural variations may also have been performed to the present peptides as referred to herein, such as synthetic modifications. Said “variant” still maintains a biological activity similar to oxytocin and said oxytocin variant is also stabilized by being present in a topical composition according to the present disclosure.

Furthermore, a “fragment” of oxytocin, as referred to herein is a peptide which comprises a part of the amino acid sequence of oxytocin, but wherein one or more amino acids may have been removed from one or both of the amino acid terminal end(s). This term also refers to a fragment of a oxytocin variant as defined in SEQ ID NO:2, hence meaning that also encompassed by the present disclosure is any fragment of a peptide as presented by SEQ ID NO:2.

Herein, “endogenous oxytocin production”, refers to the endogenous production in a subject, such as a mammal, e.g. a human, of oxytocin. Oxytocin is produced in neurons in the supraoptical nucleus (SON) and the paraventricular nucleus (PVN), in the hypothalamus and is released to the blood via the posterior pituitary. This oxytocin originates from the magnocellular cells in PVN and SON. Oxytocin is also released into important regulatory sites of the brain from axoncollaterals originating from the PVN and from oxytocinergic nerves originating from parvocellular nerons.

An “imbalance” in oxytocin production refers to a condition in a subject, such as a mammal, where the endogenous production of oxytocin is somewhat disturbed, low, insufficient and/or deficient in any manner as e.g. due to the presence of less efficient variants of oxytocin receptors so that the subject may experience a disharmony in life, e.g. depression, mental illness etc., and as further explained herein. Further, such a condition/imbalance may for example be evaluated as explained in the experimental section.

A “topical composition” as defined herein, refers to a pharmaceutical composition which is formulated to be administered topically, e.g. to any epithelial membrane, such as the vaginal epithelial membrane, then e.g. being referred to as a vaginal composition or a composition which is to be administered vaginally. The term topical composition also comprises a composition to be administered anally, then e.g. being referred to as an anal composition or a composition which is to be administered anally. The term topical composition also comprises a composition to be administered epicutaneously, then e.g. being referred to as an epicutaneous composition or a composition which is to be administered epicutaneously. Said topical composition may also be administered e.g. to the skin (epicutaneously), anally e.g. by suppositories etc. The composition may also be administered sublingually or buccally, then being referred to as a sublingual or buccal composition. This may also be referred to as a pharmaceutical composition for epicutaneous, sublingual, buccal, vaginal or anal administration. Such a topical composition may comprise in addition to oxytocin and/or a fragment or a variant thereof as defined herein, a suitable carrier and/or stabilizer, such as a gel, which is exemplified herein, making it suitable for topical administration, pH regulating entities, such as a buffer, preservatives, such as benzoic acid, and/or water producing an aqueous composition.

A “pH regulating agent” is any agent, such as a liquid agent, such as an aqueous liquid, which is able to regulate and/or maintain the pH of said pharmaceutical composition, wherein said pH is kept approximately in a selected range, which selected range is exemplified herein. Such a pH regulating agent can for example be a buffer, such as a citrate, lactate or phosphate buffer. A “buffer” is an ionic compound, usually a salt of a weak acid or base, added to a solution to resist changes in its acidity or alkalinity and thus stabilize its pH. A buffer solution is a solution containing such a compound. Other examples of a pH regulating agents are organic and inorganic acids and bases, such as acetic acid, citric acid, phosphoric acid, hydrochloric acid and sodium hydroxide.

By “disorder which is responsive to modulation of the endogenous oxytocin production” and the like is in the present context intended a disorder (pathological condition) which may be positively affected by modulation of the endogenous oxytocin production, i.e. by an increase and/or normalization in the amount of endogenously produced oxytocin. Examples of such conditions include, but are not limited to oxytocin deficiency, low social ability, anxiety, low energy level, stress sensitivity, mental climacteric symptoms, obsessive-compulsive disorder (OCD), borderline, attachment disorders, depression, autism and other forms of mental fragility or illness, pain, inflammation, an abusive disorder and/or disturbances of cardiovascular and gastrointestinal function of central origin. The disorder may benefit from an increase in endogenous oxytocin production towards more normal amounts of oxytocin, a normalization of the amount of endogenously produced oxytocin or an increase above this level, in the latter case in particular when the disorder is caused by a defect in the oxytocin receptor causing a reduced oxytocin function as further discussed elsewhere herein.

By “abusive disorders” is in the context of the present document intended disorders involving an abuse of an activity or an abuse by ingestion e.g. such as alcoholism, drug abuse, smoking, sex abuse, an eating disorder. By “eating disorder” is in the context of the present document intended anorexia or bulimia.

An “oxytocin deficiency” is in the context of the present document a deficiency which is due to either a too low amount of endogenous oxytocin being produced or due to a deficient function of the oxytocin receptor. In the latter case, the amount of oxytocin that is produced may be normal but due to a defect in the oxytocin receptor, the sensitivity of the oxytocin receptor to oxytocin is reduced. A defect in the oxytocin receptor causing a lowered oxytocin sensitivity may be due to an innate deficiency of the function of the oxytocin receptor or by a down-regulation of the oxytocin receptor function as a consequence of hormonal changes with age, such as by a lowering of oestrogen levels during menopause.

One international unit (IU) of oxytocin is the equivalent of about 1.7 μg (about 1.67 μg) of pure oxytocin peptide.

### BRIEF DESCRIPTION OF THE FIGURES

FIG. 1 shows the reduction of the scores of vaginal atrophy as measured by histological analysis of vaginal biopsies in response to a 7 week long treatment with placebo and 100 and 400 IU of oxytocin. The difference between values obtained at 1 and 7 weeks of treatment after administration of 100 and 400 IU was statistically significant, but not that for placebo.

FIG. 2 shows increase plasma levels in response to placebo, 100 and 400 IU administered intravaginally. The first value is taken before the treatment of intravaginal oxytocin has started. The following values show that oxytocin levels at 15, 30, 60 and 90 minutes after administration of oxytocin are elevated in a pulse like fashion. Note that the value obtained 10 hours after treatment are higher than those observed before treatment was started. The y-axis displays the oxytocin levels in pg/ml plasma.

FIG. 3 illustrates the women's evaluation of their most bothersome symptom. The women were asked to name which symptom that bothered them the most (dyspareunia, dysuria, vaginal dryness and itching or irritation) when they were included in the study. At the subsequent visits at 2 and 7 weeks, they were asked whether this symptom was still present or not. The size of the bars indicate the percentage of women experiencing the symptom identified as the most bothersome at the start of the study at the different time points.

FIG. 4 illustrates how much oxytocin in a citrate buffered water solution containing 4000 IU/g oxytocin has penetrated the test skin membrane into the recipient solution.

FIG. 5 shows a chromatogram of the content of oxytocin in the recipient solution that has penetrated the test skin membrane, from a buffered water solution containing 4000 IU/g oxytocin, after 48 h.

### DETAILED DESCRIPTION

The present document is based on the surprising finding that administration of oxytocin resulting in a specific blood level of oxytocin may be used for the treatment and/or prevention of a disorder which is responsive to modulation of the endogenous oxytocin production. In accordance with the present document there is therefore provided means for treating and/or preventing different disorders which are responsive to and benefit from modulation of the endogenous oxytocin production by administrating oxytocin.

The amount of oxytocin produced in a healthy object is regulated by a feed-back mechanism regulating the amount oxytocin produced. Exogenously administered oxytocin, such as by intravenous administration of oxytocin, may affect this feed-back mechanism, leading to a lower amount of endogenous oxytocin being produced, which is of course disadvantageous. Administration of oxytocin in accordance with the present disclosure appears to avoid such negative feedback inhibition of the endogenous oxytocin system. Without wishing to be bound by theory, this may be due to a stimulating effect on the endogenous oxytocin production when oxytocin is administered to a subject in accordance with the present document, without a concomitant negative feed-back inhibition on such production.

The positive effects of oxytocin when administered in accordance with the present disclosure therefore appears to occur due to a stimulating effect on endogenous oxytocin production, rather than a direct effect on a disorder by the exogenously administered oxytocin. Without wishing to be bound by theory, it is envisaged that these effects are not produced by oxytocin from the blood entering into the brain, as the blood brain barrier comes in between, but instead that oxytocin in the circulation affects and increases the activity of certain sensory nerves which in turn leads to a stimulation of the release of oxytocin from the neurons in the SON and PVN in the hypothalamus. Thereby, there is a certain increase of the oxytocin in the blood and also an enhanced release of oxytocin from the oxytocin containing neurons in the brain, which in turn creates positive effects of a disorder which is responsive to modulation of the endogenous oxytocin production. Accordingly, again without being bound by theory, this may be a bidirectional connection between peripheral and central nervous oxytocin release. Hence, circulating oxytocin may via activation of the sensory nervous system make it possible to increase the central release of endogenous oxytocin via the blood. This may be done by small, short increases of the level of oxytocin in the blood according to a breast feeding model, or alternatively by a small sustained increase of oxytocin.

Administration of oxytocin in accordance with the present document leads to an increase in the endogenous production of oxytocin. This increase may lead to endogenous oxytocin levels approaching normal endogenous oxytocin levels or a level above normal endogenous oxytocin levels. The latter is particularly beneficial in the case the disorder is caused by a defect in the oxytocin receptor, which defect leads to a lowered oxytocin sensitivity, thus demanding higher oxytocin concentrations in order for the receptor to become activated. Such disorders may therefore benefit from a higher than normal level of endogenous oxytocin.

Only 1% to 1% of oxytocin administered intravenously passes over the blood brain barrier into the brain. In order to reach the brain, when oxytocin is administered via the epithelial membrane it would therefore have to pass two barriers, first the epithelial membrane and then the blood brain barrier, and the amount of exogenously administered oxytocin reaching the brain would be very low, the amount of oxytocin reaching the brain being only ca 1/10.000 the amount of oxytocin originally exogenously administered. However, it was surprisingly found that by administration of oxytocin in accordance with the present document, it is possible to achieve the desired effects with a dose that is based on only the first dilution taking place upon passing the first barrier. Accordingly, administration of oxytocin in accordance with the present document allows a significantly lower dose of oxytocin to be administered by still allowing the provision of the same or substantially the same effect as the dose based on passing two barriers. While not wishing to be bound by any specific theory, administration of oxytocin in accordance with the present disclosure is believed to achieve the desired effect by activation of nerves via the blood and/or through a communication between the peripheral or central nerves via the blood.

Disorders

Disorders which may be treated and/or prevented in accordance with the present document include conditions which are responsive to modulation of endogenous oxytocin production.

Objects to be treated with oxytocin in accordance with the present document are in particular objects which have a deficiency the production of endogenous oxytocin, leading to an oxytocin deficiency. Such objects benefit from administration of oxytocin as disclosed herein as this results in an increase in the blood concentration of oxytocin by an increased endogenous production of oxytocin, without a concomitant inhibition of the endogenous oxytocin production. As mentioned above, one problem with previous methods involving an attempt to increase the blood concentration of oxytocin is that such an increase is often followed by a negative feed-back mechanism which causes a decrease in the endogenous oxytocin production. Even though such treatments may have a positive effect on different conditions benefiting from the administration of oxytocin, such treatments may therefore have a negative effect on the endogenous oxytocin production, thus requiring continuous administration of higher doses of oxytocin. These methods therefore provide their positive effect by the exogenously administered oxytocin rather than by causing an effect on the endogenous oxytocin production. By the administration of oxytocin as disclosed herein, the endogenous production of oxytocin is positively affected in itself and can thus be used to treat and/or prevent the oxytocin deficiency in itself, rather than masking the symptoms of such an oxytocin deficiency by exogenously administered oxytocin.

Objects benefiting from administration of oxytocin in accordance with the present disclosure are also objects which may have a normal or close to normal endogenous production of oxytocin, but which have a defect in the oxytocin receptor which leads to a lower oxytocin sensitivity. Such objects also benefit from an increase in oxytocin production as more circulating oxytocin is required to get a proper response from the oxytocin receptor.

In particular, disorders which benefit from administration of oxytocin in accordance are with the present document are selected from the group consisting of oxytocin deficiency, low social ability, anxiety, low energy level, stress sensitivity, mental climacteric symptoms, obsessive-compulsive disorder (OCD), borderline, attachment disorders, depression, autism and other forms of mental fragility or illness, pain, inflammation, an abusive disorder and/or disturbances of cardiovascular and gastrointestinal function of central origin. A disorder which in particular may be treated and/or prevented in accordance with the present document is an oxytocin deficiency. Such an oxytocin deficiency may in turn result in a disorder selected from the group consisting of low social ability, anxiety, low energy level, stress sensitivity, mental climacteric symptoms, obsessive-compulsive disorder (OCD), borderline, attachment disorders, depression, autism and other forms of mental fragility or illness, pain, inflammation, an abusive disorder and/or disturbances of cardiovascular and gastrointestinal function of central origin which may then be prevented and/or treated by the administration of oxytocin in accordance with the present document. By “abusive disorders” is in the context of the present document intended disorders involving an abuse of an activity or an abuse by ingestion e.g. such as alcoholism, drug abuse, smoking, sex abuse, an eating disorder. By “eating disorder” is in the context of the present document intended anorexia or bulimia.

Further, administration of oxytocin in accordance with the present document may also be used for effecting a local effect of oxytocin, such as wound healing, and/or treating and/or preventing an atrophic condition in the skin or mucosa, such as vaginal atrophy, depending on the site where the oxytocin is administered such as in a combination treatment with one or more of the other disorders disclosed herein.

Consequently, the present document is also directed to a method for the treatment and/or prevention of a disorder which is responsive to modulation of the endogenous oxytocin production in a subject comprising administrating a therapeutically effective amount of a composition comprising oxytocin and/or one or more fragment(s) and/or variant(s) thereof to said subject in need thereof. Further, the present document is directed to a pharmaceutical composition comprising oxytocin and/or one or more fragment(s) and/or variant(s) thereof, for use in the treatment and/or prevention of a disorder which is responsive to modulation of the endogenous oxytocin production. The present document is also directed to the use of oxytocin and/or one or more fragment(s) and/or variant(s) thereof, in the manufacture of a composition for the treatment and/or prevention of a disorder which is responsive to modulation of the endogenous oxytocin production.

Further disorders/conditions which may be treated and/or prevented by the administration of oxytocin in accordance with the present document are disclosed elsewhere herein.

The subjects which may benefit from an administration of oxytocin in accordance with the present document are, for instance, subjects that have a too low endogenous production of oxytocin, subjects which have a normal endogenous production of oxytocin but are affected or in risk of being affected by the herein disclosed disorders, or subjects which have a defect in the oxytocin receptor causing a lowered oxytocin sensitivity. A defect in the oxytocin receptor causing a lowered oxytocin sensitivity may be due to an innate deficiency of the function of the oxytocin receptor or by a down-regulation of the oxytocin receptor function as a consequence of hormonal changes with age, such as by a lowering of oestrogen levels during menopause.

Administration of the Compositions

Administration of oxytocin in accordance with the present document may take place by topical administration, such as by epicutaneous, sublingual, buccal (e.g. intrabuccal), vaginal, or anal administration. Thus, administration takes place so that the oxytocin penetrates the epithelial membrane. The oxytocin by such administration enters the blood circulation and therefore does not enter the CNS (central nervous system) as is the case when a nasal spray is used for administering oxytocin. As mentioned above, it is envisaged that the positive effects of the oxytocin when administered in accordance with the present document are not produced by oxytocin from the blood entering into the brain, as the blood brain barrier comes in between, but instead that oxytocin in the circulation affects and increases the activity of certain sensory nerves which in turn leads to a stimulation of the release of oxytocin from the neurons in the SON and PVN in the hypothalamus. Neither it is envisaged that the exogenously administered oxytocin exerts its effects by the direct effect of increasing the blood level of exogenous oxytocin, but rather that the exogenous oxytocin causes an indirect increase of the level of the endogenously produced circulating amount of oxytocin by affecting the production of oxytocin.

The oxytocin may be administered once daily, even if other administration intervals, such as twice a day, every second day, 2-3 times a week or once weekly also may be used.

By vaginal or anal administration of oxytocin in accordance with the present document, it can be expected that about 10% of the exogenously administered oxytocin is expected to be taken up (i.e. passes the vaginal or anal epithelial membrane) and enter the blood. When oxytocin is administered epicutaneously, sublingually or buccally, about 1% of the amount of exogenously administered oxytocin is expected to be taken up and enter the blood.

In order to achieve the required blood levels of exogenously administered oxytocin in order to affect the endogenous oxytocin production as desired in the present document, doses of about 10-10 000 IU of oxytocin may be administered when topical administration is used. When oxytocin is administered epicutaneously, sublingually or buccally, a dose of about 100-6 000 IU, such as about 1 000-6 000 IU or about 2 000-4 000 IU, is typical. A composition comprising about 1-15 mg/ml of oxytocin, such as about 1.7-10 mg/ml, about 1.7-6.8 mg/ml or about 3.4-6.8 mg/ml oxytocin, may be suitable for such epicutaneous, sublingual or buccal administration. When oxytocin is administered vaginally or anally, a dose of about 10-600 IU, such as about 100-600 IU or about 200-400 IU, is typical due to a higher expected uptake of oxytocin when vaginally/anally administered as compared to when epicutaneously administered. A composition comprising about 0.1-2 mg/ml of oxytocin, such as about 0.17-1 mg/ml, about 0.17-0.68 mg/ml or about 0.34-0.68 mg/ml oxytocin, may be suitable for such vaginal/anal administration. As mentioned elsewhere herein, 1 IU oxytocin corresponds to about 1.7 μg oxytocin.

As mentioned elsewhere herein, when oxytocin is administered epicutaneously, sublingually or buccally in accordance with the present document, about 1% of the administered dose of oxytocin is expected to penetrate the epithelium and enter the blood. An epicutaneously, sublingually or buccally administered dose of 10-10 000 IU oxytocin is thus expected to result in about 0.1-100 IU (corresponding to about 0.17-170 μg) oxytocin entering the blood. An epicutaneously, sublingually or buccally administered dose of oxytocin of about 100-6 000 IU, such as about 1 000-6 000 IU or about 2 000-4 000 IU, is consequently expected to result in about 1-60 IU (about 1.7-102 μg), about 10-60 IU (about 17-102 μg) or about 20-40 IU (about 34-68 μg), respectively, oxytocin entering the blood.

As mentioned elsewhere herein, when oxytocin is administered vaginally or anally in accordance with the present document, about 10% of the administered dose of oxytocin is expected to penetrate the epithelium and enter the blood. A vaginally or anally administered dose of 10-10 000 IU oxytocin is thus expected to result in about 1-1 000 IU (corresponding to about 1.7-1700 μg) oxytocin entering the blood. A vaginally or anally administered dose of oxytocin of about 10-600 IU, such as about 100-600 IU or about 200-400 IU, is consequently expected to result in about 1-60 IU (about 1.7-102 μg), about 10-60 IU (about 17-102 μg) or about 20-40 IU (about 34-68 μg), respectively, oxytocin entering the blood.

Administration of an oxytocin may in certain aspects be facilitated by applying touch, pressure, massage, heat, or infrared light on the skin, which leads to enhanced skin permeability. Hirvonen, J., Kalia, Y N, and Gay, R H. Transdermal delivery of peptides by iontophoresis. Nat Biotechnol December 1996; 14(13): 1710-1713 describes how to enhance the transport of a drug via the skin using the driving force of an applied electric field. Iontophoresis may be effected at a slightly basic pH.

The subject to which the oxytocin is administered to may be a mammal, such as a human being.

Compositions Comprising Oxytocin

The composition comprising oxytocin which is used in the context of the present document for the treatment and/or prevention of a disorder which is responsive to modulation of the endogenous oxytocin production is a (pharmaceutical) composition comprising oxytocin and/or one or more fragment(s) and/or variant(s) thereof, as well as pharmaceutically acceptable salts thereof.

The fragment(s) and/or variant(s) of oxytocin may correspond to the peptide of;

(SEQ ID NO: 2)
X₁-X₂-X₃-X₄-Asn-Cys-X₅-X₆-X₇-X₈-NH₂

wherein X₁ is selected from the group consisting of Cys and nothing;
X₂ is selected from the group consisting of Tyr, Phe, and nothing;
X₃ is selected from the group consisting of Ile, Val, Hoph, Phe, Cha, and nothing;
X₄ is selected from the group consisting of Gln, Ser, Thr, Cit, Arg, and Daba;
X₅ is selected from the group consisting of Pro and nothing;
X₆ is selected from the group consisting of Ile, Leu, nothing, Val, Hos, Daba, Thr, Arg, and Cit;
X₇ is selected from the group consisting of Gly, nothing, and Ala;
X₈ is selected from the group consisting of Gly and nothing.

In particular, fragment(s) and/or variant(s) of oxytocin may be selected from the group consisting of the peptides corresponding to SEQ ID NO:3-24.

In one aspect, when X₁ is Cys then a disulfide is formed between X₁ and Cys. Accordingly, it is to be understood that when X₁ in formula (I) is cysteine (Cys) then the thiol group of X₁ may form a disulfide with the thiol group of the cystein that is located between the asparagine (Asn) and X₅ thereby forming a cyclic structure of formula (Ia):

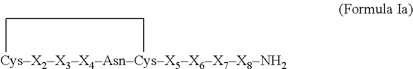

Herein, said topical composition may comprise oxytocin, i.e. when X₁ is Cys, X₂ is Tyr, X₃ is Ile, X₄ is Gln, X₅ is Pro, X₆ is Leu, X₇ is Gly, and X₈ is nothing in SEQ ID NO:2, (Cys-Tyr-Ile-Gln-Asn-Cys-Pro-Leu-Gly-NH₂) (SEQ ID NO:1).

The one or more variant(s) and/or fragment(s) of oxytocin of said topical composition (SEQ ID NO:2) may also be selected from the group consisting of the following compounds, as well as pharmaceutically acceptable salts thereof:

Mesotocin: Cys-Tyr-Ile-Gln-Asn-Cys-Pro-Ile-Gly-NH₂ (SEQ ID NO:3)

X₁ is Cys, X₂ is Tyr, X₃ is Ile, X₄ is Gln, X₅ is Pro, X₆ is Ile, X₇ is Gly, and X₈ is nothing;

Isotocin: Cys-Tyr-Ile-Ser-Asn-Cys-Pro-Ile-Gly-NH₂ (SEQ ID NO:4)

X₁ is Cys, X₂ is Tyr, X₃ is Ile, X₄ is Ser, X₅ is Pro, X₆ is Ile, X₇ is Gly, and X₈ is nothing;

Annetocin: Cys-Phe-Val-Arg-Asn-Cys-Pro-Thr-Gly-NH₂ (SEQ ID NO:5)

X₁ is Cys, X₂ is Phe, X₃ is Val, X₄ is Arg, X₅ is Pro, X₆ is Thr, X₇ is Gly, and X₈ is nothing;

Vasotocin: Cys-Tyr-Ile-Gln-Asn-Cys-Pro-Arg-Gly-NH₂ (SEQ ID NO:6)

X₁ is Cys, X₂ is Tyr, X₃ is Ile, X₄ is Gln, X₅ is Pro, X₆ is Arg, X₇ is Gly, and X₈ is nothing;

Vasopressin: Cys-Tyr-Phe-Gln-Asn-Cys-Pro-Arg-Gly-NH₂ (SEQ ID NO:7)

X₁ is Cys, X₂ is Tyr, X₃ is Phe, X₄ is Gln, X₅ is Pro, X₆ is Arg, X₇ is Gly, and X₈ is nothing;

### Cys-Tyr-Ile-Gln-Asn-Cys-NH₂ (SEQ ID NO:8)

X₁ is Cys, X₂ is Tyr, X₃ is Ile, X₄ is Gln, and X₅-X₈ is nothing;

### Cys-Tyr-Ile-Gln-Asn-Cys-Pro-NH₂ (SEQ ID NO:9)

X₁ is Cys, X₂ is Tyr, X₃ is Ile, X₄ is Gln, X₅ is Pro, and X₆-X₈ is nothing;

### Cys-Tyr-Ile-Gln-Asn-Cys-Pro-Leu-NH₂ (SEQ ID NO:10)

X₁ is Cys, X₂ is Tyr, X₃ is Ile, X₄ is Gln, X₅ is Pro, X₆ is Leu, and X₇₋₈ is nothing;

### Tyr-Ile-Gln-Asn-Cys-Pro-Leu-Gly-NH₂ (SEQ ID NO:11)

X₁ is nothing, X₂ is Tyr, X₃ is Ile, X₄ is Gln, X₅ is Pro, X₆ is Leu, X₇ is Gly, and X₈ is nothing;

### Ile-Gln-Asn-Cys-Pro-Leu-Gly-NH₂ (SEQ ID NO:12)

X₁-X₂ is nothing, X₃ is Ile, X₄ is Gln, X₅ is Pro, X₆ is Leu, X₇ is Gly, and X₈ is nothing;

### Gln-Asn-Cys-Pro-Leu-Gly-NH₂ (SEQ ID NO:13)

X₁-X₃ is nothing, X₄ is Gln, X₅ is Pro, X₆ is Leu, X₇ is Gly, and X₈ is nothing;

### Ile-Gln-Asn-Cys-Pro-NH₂ (SEQ ID NO:14)

X₁-X₂ is nothing, X₃ is Ile, X₄ is Gln, X₅ is Pro, and X₆-X₈ is nothing;

### Cys-Tyr-Ile-Gln-Asn-Cys-Pro-Leu-Gly-Gly-NH₂ (SEQ ID NO:15)

X₁ is Cys, X₂ is Tyr, X₃ is Ile, X₄ is Gln, X₅ is Pro, X₆ is Leu, X₇ is Gly, and X₈ is Gly;

### Gln-Asn-Cys-Pro-Leu-Leu-NH₂ (SEQ ID NO:16)

X₁-X₃ is nothing, X₄ is Gln, X₅ is Pro, X₆ is Leu, X₇ is Leu, and X₈ is nothing;

### Cys-Tyr-Val-Thr-Asn-Cys-Pro-Leu-Gly-NH₂ (SEQ ID NO:17)

X₁ is Cys, X₂ is Tyr, X₃ is Val, X₄ is Thr, X₅ is Pro, X₆ is Leu, X₇ is Gly, and X₈ is nothing;

### Cys-Tyr-Hoph-Thr-Asn-Cys-Pro-Val-Gly-NH₂ (SEQ ID NO:18)

X₁ is Cys, X₂ is Tyr, X₃ is Hoph, X₄ is Thr, X₅ is Pro, X₆ is Val, X₇ is Gly, X₈ is nothing;

### Cys-Tyr-Phe-Cit-Asn-Cys-Pro-Leu-Gly-NH₂ (SEQ ID NO:19)

X₁ is Cys, X₂ is Tyr, X₃ is Phe, X₄ is Cit, X₅ is Pro, X₆ is Leu, X₇ is Gly, and X₈ is nothing;

### Cys-Tyr-Cha-Arg-Asn-Cys-Pro-Hos-Ala-NH₂ (SEQ ID NO:20)

X₁ is Cys, X₂ is Tyr, X₃ is Cha, X₄ is Arg, X₅ is Pro, X₆ is Hos, X₇ is Ala, and X₈ is nothing;

### Cys-Tyr-Val-Daba-Asn-Cys-Pro-Daba-Ala-NH₂ (SEQ ID NO:21)

X₁ is Cys, X₂ is Tyr, X₃ is Val, X₄ is Daba, X₅ is Pro, X₆ is Cit, X₇ is Ala, and X₈ is nothing;

### Cys-Tyr-Hoph-Daba-Asn-Cys-Pro-Cit-Ala-NH₂ (SEQ ID NO:22)

X₁ is Cys, X₂ is Tyr, X₃ is Hoph, X₄ is Daba, X₅ is Pro, X₆ is Cit, X₇ is Ala, and X₈ is nothing;

### Cys-Tyr-Phe-Arg-Asn-Cys-Pro-Val-Ala-NH₂ (SEQ ID NO:23)

X₁ is Cys, X₂ is Tyr, X₃ is Phe, X₄ is Arg, X₅ is Pro, X₆ is Val, X₇ is Ala, and X₈ is nothing; and

### Cys-Tyr-Cha-Cit-Asn-Cys-Pro-Arg-Gly-NH₂ (SEQ ID NO:24)

X₁ is Cys, X₂ is Tyr, X₃ is Cha, X₄ is Cit, X₅ is Pro, X₆ is Arg, X₇ is Gly, X₈ is nothing;

Accordingly, there is provided herein a topical composition, wherein said one or more fragment(s) and/or variant(s) of oxytocin is/are selected from the group consisting of the peptides corresponding to SEQ ID NO:3-SEQ ID NO:24.

The unnatural amino acids in said substances have the following structures:

Cyclohexylalanine, herein referred to as Cha,

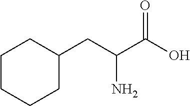 [SMILES: NC(CC1CCCCC1)C(=O)O]

Homophenylalanine, herein referred to as Hoph,

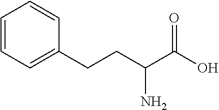 [SMILES: NC(CCc1ccccc1)C(=O)O]

Citrulline, herein referred to as Cit,

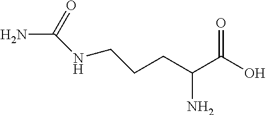 [SMILES: NC(=O)NCCCC(N)C(=O)O]

Diaminobutyric acid, herein referred to as Daba, and

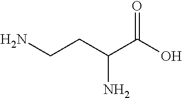

Homoserine, herein referred to as Hos,

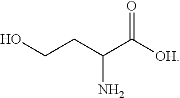

When a position in SEQ ID NO:2 is stated as being “nothing” it means that it represents a single bond between the items (letter, atom or group).

Other variants of oxytocin could also be used in compositions as provided herein, such as naturally occurring or artificially modified variants, analogues, and/or derivatives of oxytocin, mesotocin, isotocin, and/or annetocin. Such variants could be obtained by addition, insertion, elimination, or substitution of at least one amino acid in these hormones. Other substances include precursors, metabolites such as metabolic derivatives e.g. metabolic degradation products of the substances mentioned herein displaying the same properties.

Metabolic derivatives or metabolic degradation products may be oxytocin like peptides, e.g. with nine amino acids such as oxytocin, mesotocin, isotocin, and annetocin from which one or more amino acids has been deleted from either the carboxyl terminal end or the amino terminal end or both the carboxyl terminal and the amino terminal end, such as 1-3 amino acids from each terminal. In certain aspects, one, two or three amino acids may have been deleted from the carboxy terminal end i. e. Gly only, Gly and Leu, or Gly, Leu, and Pro. Preferably one, two or three amino may have been deleted from the amino terminal i. e. Cys only, Cys and Tyr, or Cys, Tyr, and Ile.

In certain aspects one, two or three amino acids may have been deleted both from carboxy terminal end i. e. Gly only, Gly and Leu, or Gly, Leu, and Pro, and one, two or three amino acids may have been deleted from the amino end i. e. Cys only, Cys and Tyr, or Cys, Tyr, and Ile. It could be ascertained that these variants are analogues of oxytocin, mesotocin, isotocin or annetocin by immunological methods, e.g. RIA (radioimmunoassay), IRMA (radiometric methods), RIST (radioimmunosorbent test), and RAST (radioallergosorbent test). It is also included variants of oxytocin having at least 50, 60, 70, 80, 90, 95, 96, 97, 98, or 99% sequence identity to oxytocin, said variants showing an oxytocin activity, as defined herein.

Annetocin has been isolated from the earthworm, as described in Oumi T. Ukena K, Matsushima O, Ikeda T, Fujita T, Minakata H, Nomoto K, Annetocin: an oxytocin-related peptide isolated from the earthworm, Eisenia foetida, Biochem Biophys Res Commun 1994, January 14; 198(1); 393-399.

There is also a possibility to create new compounds with oxytocin activity by means of computer simulation. Methods for computer simulation are known by a person skilled in the art, e.g. as described in EP 0660 210 A2.

There is also provided herein a topical composition comprising oxytocin and/or a variant thereof in both D- and L-form, as well as racemates thereof. In certain aspects the disclosure relates to the L-form. By inversion of the peptide sequence thereof, the D-form could be converted to the L-form. These and the peptides above can be produced by methods known to a person skilled in the art, e.g. according to Merrifield, P. B., “Solid Phase Synthesis”, Angew. Chemie, 1985, No. 97, p. 801.

It should be noted that pharmaceutically acceptable salts of the compounds are also provided herein. Examples of salts of the compounds are pharmaceutically acceptable acid and base addition salts.

Further, examples of salts of the compounds forming the topical compositions of the present disclosure are pharmaceutically acceptable acid and base addition salts.

The expression “pharmaceutically acceptable acid addition salts” are intended to be any non-toxic organic or inorganic acid addition salt of the compounds of SEQ ID NO: 2, and/or any other variants and/or fragments of oxytocin as described herein. Examples of illustrative inorganic acids that form suitable salts are hydrochloric acid, hydrobromic acid, sulphuric acid, phosphoric acid and acid metal salts such as sodium monohydrogen ortophosphate and potassium hydrogensulphate. Examples of illustrative organic acids that form suitable salts are mono-, di- and tricarboxylic acids. Examples of such acids are acetic acid, glycolic acid, lactic acid, pyruvic acid, malonic acid, succinic acid, glutaric acid, fumaric acid, malic acid, tartaric acid, citric acid, ascorbic acid, maleic acid, hydroxymaleic acid, benzoic acid, hydroxybenzoic acid, phenylacetic acid, cinnamic acid, salicylic acid, 2-phenoxybenzoic acid, and sulphonic acids such as p-toluenesulphonic acid, metha-nesulphonic acid and 2-hydroxyethanesulphonic acid. Such salts could either be in hydrated or anhydrous form. The acid addition salts of these compounds are generally water soluble and different hydrophilic organic solvents and, that compared to the free base forms thereof, generally display higher melting points.

The expression “pharmaceutically acceptable base addition salts” are intended to be any non-toxic organic or inorganic base addition salt of the compounds of SEQ ID NO: 2, and/or any other variants and/or fragments of oxytocin as described herein. Examples of illustrative inorganic bases that form suitable salts are alkali and earth alkali metal hydroxides and carbonates such as sodium hydroxide, sodium carbonate, potassium hydroxide, potassium carbonate, calcium hydroxide, calcium carbonate, magnesium hydroxide, magnesium carbonate and ammonia. Examples of illustrative organic bases that form suitable salts are methylamine, dimethylamine, trimethylamine and picoline. Either mono- or dibasic salts could be formed with such compounds. The base addition salts of these compounds are generally water soluble and different hydrophilic organic solvents and, that compared to the free base forms thereof, generally display higher melting points.

The composition comprising oxytocin may be a topical composition, such as an epicutaneous, sublingal, buccal, vaginal or anal composition. Such topical compositions enable an uptake of oxytocin over the epithelial membrane.

The pH of the composition comprising oxytocin is typically between about 3 and 5, such as between about 3 and 4, such as between about 3 and 3.5. The pH may thus be in the range of between about 3 and 4.5, such as between about 3.5 and about 4.5, between about 3.5 and 4, between about 3 and 4, between about 3 and 3.5, between about 3 and 3.1, between about 3 and 3.2, between about 3 and 3.3, between about 3 and 3.4, between about 3 and 3.6, between about 3 and 3.7, between about 3 and 3.8, between about 3 and 3.9, between about 3.1 and 3.2, between about 3.1 and 3.3, between about 3.1 and 3.4, between about 3.1 and 3.5, between about 3.1 and 3.6, between about 3.1 and 3.7, between about 3.1 and 3.8, between about 3.1 and 3.9, between about 3.2 and 3.3, between about 3.2 and 3.4, between about 3.2 and 3.5, between about 3.2 and 3.6, between about 3.2 and 3.7, between about 3.2 and 3.8, between about 3.2 and 3.9, between about 3.3 and 3.5, between about 3.3 and 3.6, between about 3.3 and 3.7, between about 3.3 and 3.8, or between about 3.3 and 4, or about as exemplified by the separate values. It should be noted that these values are not exact, meaning that they can vary slightly around the values provided and also include the values provided. Further, it will be appreciated that the expression “between about . . . and . . . ” is equivalent to “from about . . . to about . . . ”.

The pH the composition comprising oxytocin may be regulated by adding a pH regulating agent to said composition, such as a buffer. Herein, said buffer may be a lactate buffer, a citrate buffer, a phosphate buffer, or a mixture thereof, but is not limited thereto. The concentration of a buffer to be added to the composition comprising oxytocin may be between about 20 and 100 mM, such as between about 25 mM and 100 mM, or about 25 to 50 mM, about 25 mM to 75 mM, or about 50 to 70 mM in an aqueous solution, but is not limited thereto. It should be noted that these values are not exact, meaning that they can vary slightly around the values provided. Depending on which pH is required and which buffer is used in a topical composition, the concentration of the buffer may vary in accordance with the above.

The composition comprising oxytocin may further comprise one or more epithelium penetration enhancer(s). Such an epithelium penetration enhancer may increase the amount of oxytocin which penetrates the epithelium, such as the epicutaneous, sublingual, buccal, vaginal or anal epithelium. Examples of such epithelium penetration enhancers include, but are not limited to propylene glycol (PG), SDS, isopropyl myristate, ethanol, diethylene glycol ethyl ether and/or cineole.

The composition comprising oxytocin may further comprise a carrier e.g. in the form of a non-ionic cellulose ether. Cellulose ethers are named after, and based on, cellulose which is a renewable material and the most common chemical compound in organic nature. There is a broad range of cellulose ethers available on the market, both ionic and non-ionic, for example sodium carboxymethylcellulose, hydroxyethylethylcellulose, hydroxyethylcellulose, hydroxypropylcellulose, hydroxyethylmethylcellulose and hydroxypropylmethycellulose.

Cellulose ethers are used as additives in such diverse applications as food, paint, oil recovery, paper, cosmetics, pharmaceuticals, adhesives, printing, agriculture, ceramics, textiles, detergents and building materials. Cellulose ethers improve the product quality in these applications and act as thickeners, water retention agents, suspending aids, protecting colloids, film formers or thermoplastics in such different products as dispersion paints, drilling muds, ice cream, tablet coatings, wallpaper paste and tile adhesive.

The cellulose ethers may be non-ionic, wherein alkyl and/or hydroxyalkyl groups are attached to the anhydroglucose units by ether linkages, which form hydroxyalkylalkylcelluloses, wherein the alkyl groups have from 1 to 4 carbon atoms. A non-ionic cellulose ether is based on cellulose, which has been chemically modified in order to attain solubility in water by substitution of various groups onto the cellulose backbone. The substituents are characterised to have no electric charge when dissolved in water at a neutral pH. Non-ionic cellulose ethers such as methylcellulose, hydroxypropylmethylcellulose (also referred to as hypromellose) and methylhydroxyethylcellulose, are widely used in the pharmaceutical industry due to their ability to thicken, bind and retain water, as well as to emulsify and suspend particles and form films.

Further information regarding non-ionic cellulose ethers can be found e.g. in WO92/09307.

The properties of the non-ionic cellulose ethers are determined by the molecular weight, (e.g. the degree of polymerisation), the type of substituents and also by the number and distribution of the substituents along the molecule. Hence, depending on the oxytocin and/or fragment and/or variant thereof which is present in the composition, the non-ionic cellulose ether can have varying properties and will be determined according to the prevailing circumstances.

An example of representative cellulose ethers which may be used in a topical composition hereom are methyl cellulose (MC), hydroxyethylmethyl cellulose (HEMC), hydroxypropylmethylcellulose (HPMC), hydroxyethylethylcellulose (HEEC), and hydroxypropylcellulose (HPC). These polymers have substituents that are either nonpolar (e.g. methyl) or slightly polar (e.g. hydroxyethyl), which in combination with the hydrophilic cellulose backbone provide an amphiphilic polymer.

The composition comprising oxytocin may thus further comprise at least one non-ionic cellulose ether, such as methyl cellulose (MC), hydroxypropylmethylcellulose (HPMC), hydroxypropylcellulose (HPC), hydroxypropylmethylcellulose (HPMC), hydroxyethylethylcellulose (HEEC) and hydroxyethylmethyl cellulose (HEMC). In particular the composition comprising oxytocin may contain the non-ionic cellulose ether hydroxypropylmethylcellulose (HPMC). The composition comprising oxytocin may thus e.g. be a composition comprising oxytocin (SEQ ID NO:1) and hydroxypropylmethylcellulose (HPMC).

An exemplary composition comprising oxytocin is a composition comprising about 0.1 to 2 mg/ml, such as about 0.17-1 mg/ml, about 0.17-0.68 mg/ml or about 0.34-0.68 mg/ml, oxytocin and/or one or more fragment(s) and/or variant(s) of oxytocin and a non-ionic cellulose ether, such as hydroxypropylmethyl cellulose, said composition having a pH of about 3-5, such as about 3-4. The concentration of hydroxypropylmethyl cellulose may be about 1-5 wt %, such as about 2-4 wt % or about 3 wt %. Such a composition may further comprise an epithelium penetration enhancer, such as propylene glycol (PG), SDS, isopropyl myristate, ethanol, diethylene glycol ethyl ether and/or cineole. Such a composition is particularly suitable for vaginal or anal administration.

Another exemplary composition comprising oxytocin is a composition comprising about 1 to 15 mg/ml, such as about 1.7-10 mg/ml, about 1.7-6.8 mg/ml or about 3.4-6.8 mg/ml oxytocin and a non-ionic cellulose ether, such as hydroxypropylmethyl cellulose, said composition having a pH of about 3-5, such as about 3-4. The concentration of hydroxypropylmethyl cellulose may be about 1-5 wt %, such as about 2-4 wt % or about 3 wt %. Such a composition may further comprise an epithelium penetration enhancer, such as propylene glycol (PG), SDS, isopropyl myristate, ethanol, diethylene glycol ethyl ether and/or cineole. Such a composition is particularly suitable for epicutaneous, sublingual or buccal administration.

As will be further mentioned herein, the composition comprising oxytocin should be administered to a subject in a dose which modulates the endogenous oxytocin production. Exemplary suitable doses include a dose of about 10-10 000 IU. The dose may also be about 100-6 000 IU, about 1 000-6 000 IU or about 2 000-4 000 IU. Such doses are particularly suitable for epicutaneous, sublingual or buccal administration. The dose may also be about 10-600 IU, about 100-600 IU or about 200-400 IU. Such a dose is particularly suitable for vaginal or anal administration.

It is one object of the present document to administer a composition comprising oxytocin as defined herein to a subject in order to achieve a dosage of oxytocin entering the blood of about 15 and about 70 μg, such as between about 15 and 30 μg, 20-30 μg or between and 40 μg, such as about 20 μg (about 11.8 UI) when administered to a subject, such as by topical administration.

Further pharmaceutically acceptable carriers may be used in the context of the present disclosure, which are suitable for use in a composition comprising oxytocin.

The composition comprising oxytocin may be in the form of a gel, a suppository, a crème, a liquid suspension, a lozenge, a patch, a film etc.

One exemplary composition comprising oxytocin which may be used in the context of the present document is the Vagitocin® gel, the contents of which is disclosed elsewhere herein.

Further exemplary compositions comprising oxytocin for use in accordance with the present document are disclosed elsewhere herein.

Kit of Parts

The present document is also directed to a kit of parts comprising a composition comprising oxytocin and/or one or more fragment(s) and/or variant(s) thereof as defined elsewhere herein, a container, such as a tube, a container for a stick, an ampoule etc., for said composition, and optionally instructions for use and/or means for applying said composition to a subject. The container may be made of any suitable materials, such as plastics, metal or glass. Exemplary means for applying the composition include, but are not limited to a spatula, a syringe for exact dosing, a wipe, a device for inserting the composition (e.g. when in the form of a pill or a suppository) in the vagina or anus.

A container or dispenser may e.g. be a plastic tube or box, or a type of vessel, or a foil container etc. A kit may also comprise one or more containers each comprising one or more of a pharmaceutically acceptable carrier, adjuvant and/or excipient.

Further Aspects

The below aspects may be used in any combination with the above disclosed aspects of the present document.

There is also provided herein a composition, such as a topical composition, comprising oxytocin and/or one or more fragment(s) and/or variant(s) thereof for use in treating and/or preventing an imbalance in the endogenous production and/or function of oxytocin. Said prevention and/or treatment of the endogenous production and/or function of oxytocin comprise increasing the endogenous oxytocin production to a suitable degree as described herein. There is also provided a topical composition comprising oxytocin and/or one or more fragment(s) and/or variant(s) thereof for use in treating and/or preventing vaginal atrophy and an imbalance in the endogenous production and/or function of oxytocin. Such a topical composition provides a combination treatment and/or prevention of both vaginal atrophy and an imbalance in the endogenous production and/or function of oxytocin.

It is an object to provide uses of compositions comprising oxytocin which makes it possible to generate positive effects on the endogenous production of oxytocin. Further, there is also an objective art to identify compositions comprising oxytocin which are capable of providing both a local effect on an epithelial membrane, such as on a mucosal membrane, as well as a systemic effect thereby affecting the endogenous production and/or function of oxytocin. Such a composition could then be suitable for combination treatments for subjects suffering both from a physical and a mental disorder or discomfort, which is often experienced during menopause.

Accordingly, the present disclosure concerns a novel use of oxytocin in a topical composition, said topical composition comprising oxytocin and/or one or more fragment(s) and/or variant(s) thereof as defined herein, for use in treating and/or preventing an imbalance in the endogenous production and/or function of oxytocin. Further, such as use may also be defined as a use in treating and/or preventing a disorder caused by and/or related to a deficiency in endogenous oxytocin production and/or for use in increasing the blood plasma level of oxytocin.

There is also provided a topical composition comprising exogenous oxytocin and/or one or more fragment(s) and/or variant(s) thereof, wherein administration thereof results in an increase of circulating exogenously provided oxytocin thereby stimulating the endogenous production and release of oxytocin into the blood and/or to the brain. Hence, an imbalance, such as an underproduction of endogenous oxytocin and/or decreased function may be treated and/or prevented by raising the blood plasma concentration of oxytocin through topical administration of exogenous oxytocin and/or a fragment and/or a variant thereof as described herein.

Accordingly, there is provided herein a topical composition comprising oxytocin and/or a fragment or variant thereof for use in treating and/or preventing an imbalance in the endogenous production and/or function of oxytocin, wherein said imbalance results in a disorder, such as oxytocin deficiency, low social ability, anxiety, low energy level, stress sensitivity, mental climacteric disorders, obsessive-compulsive disorder (OCD), borderline, attachment disorders, depression autism and other forms of mental fragility or illness, as well as pain, inflammation, an abusive disorder and/or disturbances of cardiovascular and gastrointestinal function of central origin, in particular when these are due to an imbalance in the endogenous production and/or function of oxytocin. Such a composition may be administered topically, such as by vaginal administration.

A topical composition comprising oxytocin as provided herein is capable of entering the blood plasma e.g. by virtue of being administered vaginally, which absorption surprisingly occurs via the epithelial membrane, in a dosage making it suitable for efficiently regulating the endogenous production of oxytocin in an individual, such as a human being.

Accordingly, the present disclosure also relates to a topical composition which allows for oxytocin to pass through an epithelial membrane, e.g. through the vaginal membrane, making it possible to achieve sustained pulse-like increases of the plasma level of oxytocin, preferably over a long period of time. The topical mode of administration of oxytocin appears to avoid negative feedback inhibition of the endogenous oxytocin system. Thus, a topical composition as provided also herein allows for treating both a local and a systemic disorder as disclosed herein.

The present disclosure also relates to a method for treating and/or preventing an imbalance in the endogenous production and/or function of oxytocin in a subject comprising topically administrating a therapeutically effective amount of a topical composition comprising oxytocin and/or a fragment or variant thereof, as described herein, to said subject in need thereof.

The present disclosure also relates to a method for treating and/or preventing vaginal atrophy and an imbalance in the endogenous production and/or function of oxytocin in a subject comprising topically administrating a therapeutically effective amount of a topical composition comprising oxytocin and/or a fragment or variant thereof, as described herein, to said subject in need thereof.

A topical formulation comprising oxytocin of the present disclosure is capable of being absorbed through the epithelial membrane, e.g. through the vaginal membrane. Further, surprisingly, such an administration mode is believed to provide additional advantages in the form of positive effects on the endogenous production of oxytocin, which was not possible through intravenous administration of oxytocin. Accordingly, by providing accurate doses of oxytocin by a topical administration, and through the epithelial membrane, it is believed that it is possible to generate a stimulating and/or increasing effect on the endogenous production of oxytocin further providing unexpected positive effects on the central nervous system via the body's own oxytocin production and/or release providing a more sustainable prevention and/or treatment of conditions where this production/release is not in balance.

This will make such topical compositions especially useful for treating and/or preventing disorder(s) and/or condition(s) which is/are the result of and/or the cause of an imbalance of oxytocin production and/or function, and/or when the oxytocin secretion from the brain is low. Surprisingly it has been shown herein that it is possible through topical administration, e.g. by vaginal administration, to raise the blood plasma level of oxytocin and thereby possibly increase the activity of the oxytocin producing nerves in the brain. When the activity in these neurons is increased, there is an increased activity and release of endogenously produced oxytocin both to the blood and to the brain. Without wishing to be bound by any specific theory, the increased levels in the blood may be seen as a mirror of the activities in the brain which creates the positive effects of a normal balance of oxytocin in the brain.

Accordingly, there is also provided herein a topical composition comprising oxytocin for use in regulating and/or stimulating the levels, i.e. the amount and/or concentration of oxytocin in blood plasma, brain and/or the circulation as a whole, through topical, e.g. vaginal administration thereof. Hence, it is now believed that a topical composition comprising oxytocin, i.e. a composition that is to be administered topically and which is absorbed via the epithelial membrane, achieves a positive effect, via the circulation, on the endogenous production of oxytocin without activating the negative feed-back systems. Instead, the administration of oxytocin via such a route may stimulate the production of endogenous oxytocin as further explained herein.

It was surprising to find that oxytocin was taken up into blood plasma by being vaginally administered. Hence, by a topical composition as presented herein it appears possible to create a positive effect of externally administered oxytocin with no or little effect on the negative feedback mechanism of the endogenous oxytocin production in a subject. Such a topical composition provides an advantageous mode of administration by allowing epithelial uptake and facilitating the uptake of oxytocin into the blood plasma to thereby, as a secondary effect, possibly positively regulate the endogenous production of oxytocin by stimulation of the production thereof.

Accordingly, with a topical composition as described herein, without wishing to be bound by theory, appropriate doses of oxytocin may be administered to a subject in need thereof to effectively regulate a positive feedback system of oxytocin. A topical composition as described herein, for epithelial uptake, such as epicutaneous, sublingual, buccal, vaginal or anal uptake, has the ability to provide suitable concentrations of oxytocin to the circulation, affording advantageous effects on the state of mind.

Hence, the present disclosure relates to a topical composition comprising oxytocin and/or one or more fragment(s) and/or variant(s) thereof, for use in treating and/or preventing an imbalance in the endogenous production and/or function of oxytocin. Such a use may also be described as a use in treating and/or preventing a deficiency in endogenous oxytocin production and/or for use in increasing the blood plasma level of oxytocin. Such a composition may further be for use for treating and/or preventing vaginal atrophy. Said imbalance/deficiency may be related to the endogenous release of oxytocin from the brain or alternatively to less functional oxytocin receptors, thus demanding higher oxytocin concentrations in order to become activated.

The present disclosure also provides a topical composition comprising oxytocin and/or one or more fragment(s) and/or variant(s) thereof, for use in treating and/or preventing vaginal atrophy and/or an imbalance in the endogenous production and/or function of oxytocin. Such a topical composition is hence envisaged for a combination therapy for vaginal atrophy and an imbalance in the production and/or function of oxytocin. Accordingly, there is also provided herein a topical composition comprising oxytocin and/or one or more fragment(s) and/or variant(s) thereof, for use as a combination treatment and/or prevention of vaginal atrophy and an imbalance in the endogenous production and/or function of oxytocin.

Hence, it is provided herein a topical composition for use in treating and/or preventing an imbalance in the endogenous production and/or function of oxytocin, wherein said imbalance results in a disorder, such as a disorder selected from the following disorders: oxytocin deficiency anxiety, low energy level, stress sensitivity, mental climacteric disorders, decreased social ability, obsessive-compulsive disorder (OCD), borderline, autism, depression, an abusive disorder and/or other forms of mental fragility or illness. Accordingly, it is intended to treat and/or prevent a disorder as mentioned herein by virtue of influencing the endogenous production of oxytocin, by e.g. stimulation and/or regulation as described herein. Surprisingly, a topical composition may be used for such a purpose, which has not previously been shown. Said deficiency may be treated and/or prevented by stimulating the production of oxytocin into the circulation, resulting in the use in the treatment and/or prevention of disorders which are caused by and/or related to a deficiency/imbalance in the endogenous oxytocin production.

There is further provided herein a topical composition, such as a vaginal composition for use in the treatment and/or prevention of a disorder selected from oxytocin deficiency, low social ability, anxiety, low energy level, stress sensitivity, mental climacteric symptoms, obsessive-compulsive disorder (OCD), borderline, attachment disorders, depression, autism and other forms of mental fragility or illness, as well as pain, inflammation, an abusive disorder and/or disturbances of cardiovascular and gastrointestinal function of central origin. Said disorder may be caused by and/or related to an imbalance in the endogenous production and/or function of oxytocin. Such a composition may further be for use for treating and/or preventing vaginal atrophy.

There is also provided use of oxytocin and/or one or more fragment(s) and/or variant(s) thereof, in the manufacture of a topical composition for use in treating and/or preventing an imbalance in the endogenous production and/or function of oxytocin, wherein said imbalance results in a disorder selected from the group consisting of: oxytocin deficiency, low social ability, anxiety, low energy level, stress sensitivity, mental climacteric disorders, obsessive-compulsive disorder (OCD), borderline, attachment disorders, depression autism, an abusive disorder and other forms of mental fragility or illness.

There is also provided herein, a topical composition for use in treating and/or preventing an imbalance in the endogenous production and/or function of oxytocin, wherein said imbalance results in a disorder selected from the group consisting of oxytocin deficiency, low social ability, anxiety, low energy level, stress sensitivity, mental climacteric disorders, obsessive-compulsive disorder (OCD), borderline, attachment disorders, depression autism, an abusive disorder and other forms of mental fragility or illness.

There is also provided use of oxytocin and/or one or more fragment(s) and/or variant(s) thereof, in the manufacture of a topical composition for use in treating and/or preventing an imbalance in the endogenous production and/or function of oxytocin and/or in the treatment and/or prevention of vaginal atrophy, wherein said imbalance results in a disorder selected from the group consisting of: oxytocin deficiency, low social ability, anxiety, low energy level, stress sensitivity, mental climacteric disorders, obsessive-compulsive disorder (OCD), borderline, attachment disorders, depression autism, an abusive disorder and/or other forms of mental fragility or illness. In addition, the present composition provides the local effects on vaginal atrophy, expanding the use also to physical disorders, e.g. the vaginal dryness experienced during menopause.

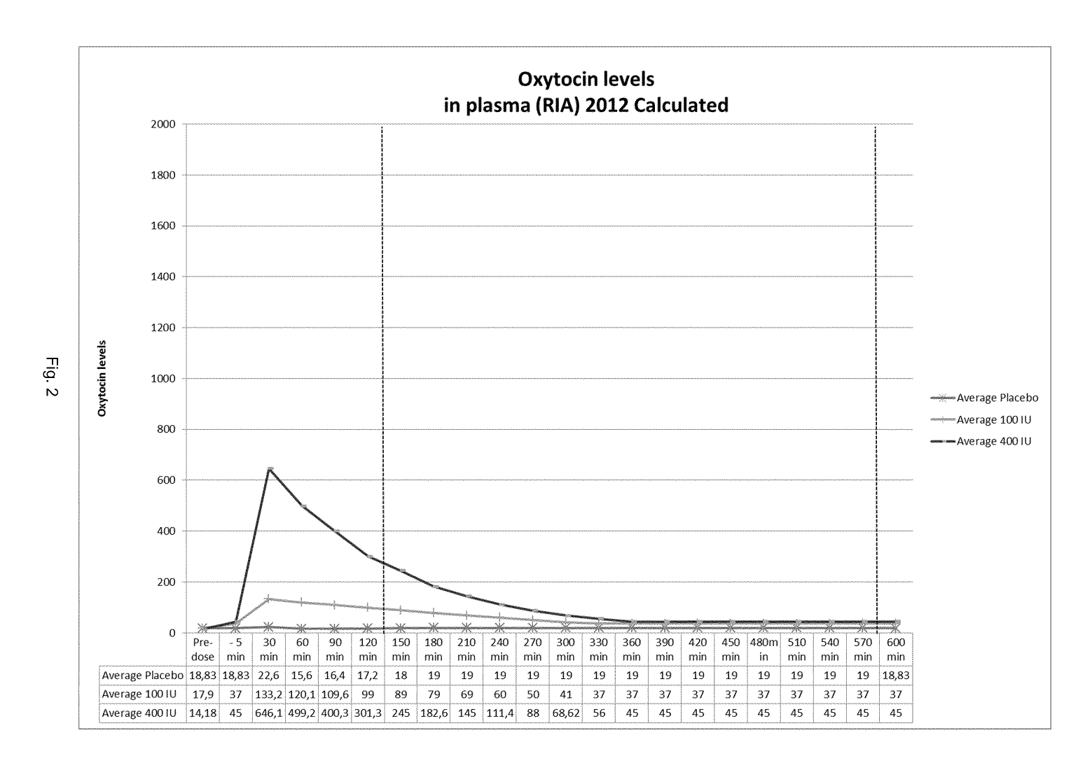

As an example, it is shown that about 10% of a topical composition of oxytocin comprising a non-ionic cellulose ether is absorbed via the epithelial membrane and further into the blood stream therein resulting in rise of plasma oxytocin levels which can be characterized as a particular area under the curve (see FIG. 2). This particular type of rise of oxytocin levels was linked to an increase of endogenous oxytocin release as reflected by a sustained increase of oxytocin levels in the plasma long after the oxytocin administered by intravaginal oxytocin was eliminated. In addition this sustained increase of peripheral oxytocin levels was paralleled by an increase of the release of neurons in the brain resulting in positive mental effects on the patient. In contrast long term intravenous administration of exogenous oxytocin which provides a long term flat immobile oxytocin curve, results in negative effects on the endogenous production of oxytocin in a subject and negative effects. Accordingly, the advantageous effects resulting from administration of a topical composition of the present disclosure were not achievable by using intravenous administration. In addition, the topical administration mode provides an effect on the central nervous system which gives a positive effect on the endogenous production of oxytocin. Further, but again not wishing to be bound by theory, the regulation appears to occur through a communication between the peripheral and the central nervous system as a consequence of the increased oxytocin levels in the circulation and/or the concentration of oxytocin in the circulation.

Intravenous administration has previously been shown to cause a negative effect on the endogenous production of oxytocin, which may be avoided by utilizing a topical administration mode instead. This opens up for additional uses of topical compositions of oxytocin to treat and/or prevent any disorders which are related to an imbalance and/or insufficiency in the endogenous oxytocin production and/or function.

Hence, by administering a topical composition of oxytocin to a subject in need thereof, for instance on a daily basis for as long as desired, it is shown herein to be possible to generate an appropriate secretion of oxytocin in the circulation to effectively regulate the endogenous feed-back mechanism of oxytocin production, i.e. to stimulate the endogenous production. This will make it possible to prevent and/or a treat disorder and/or a condition which is dependent on the endogenous level of oxytocin, and it may, as described herein, be performed by the usage of a topical composition of oxytocin instead of e.g. an intravenous administration form which is not as convenient as described herein.

Herein, such a regulation and/or stimulation of the endogenous oxytocin production in a subject is performed by a composition which may be topically administered to the peripheral blood stream, wherein said amount passing through an epithelial membrane is a dosage of about 15 and about 70 μg, such as between about 15 and 30 μg, 20-30 μg or between 15 and 40 μg, such as about 20 μg (about 11.8 UI). As an example, this is achieved by administering a topical composition comprising a dosage of oxytocin and/or a fragment or a variant thereof between about 50 and 600 IU of oxytocin, such as between about 100 and 200 UI, 100 to 300 UI, or about 50, 60, 70, 80 90, 100, 150, 200, 250, 300, 350, 400 or 500 IU, as exemplified by oxytocin in a composition comprising hydroxypropylmethylcellulose (HPMC). A topical composition for use as defined herein may be administered vaginally, i.e. it is also provided herein a topical composition for use in treating and/or preventing an imbalance in the endogenous production and/or function of oxytocin comprising oxytocin and hypromellose (HPMC), wherein said topical composition is to be administered vaginally. It is provided herein a topical composition comprising oxytocin and hydroxypropylmethylcellulose for use in the treatment and/or prevention of an imbalance in the endogenous production and/or function of oxytocin, wherein said composition is to be administered vaginally in a dosage of between about 50 and 400 IU, such as about 100 IU.

It is one object of the present document to administer a composition comprising oxytocin as defined herein to a subject in order to achieve a dosage of oxytocin entering the blood of about 15 and about 70 μg, such as between about 15 and 30 μg, 20-30 μg or between 15 and 40 μg, such as about 20 μg (about 11.8 UI) when administered to a subject, such as by topical administration.

The concentration of oxytocin and/or a fragment and/or a variant thereof as defined herein may also be between about 0.1 to 1.5 mg/ml, such as about 0.5 to about 1.5 mg/ml, about 0.5 to about 1 mg/ml, about 0.5 to about 1.2 mg/ml, about 0.2 to about 0.5 mg/ml, about 0.1 to about 0.8 mg/ml, or about 0.2 to about 1.2 mg/ml of the total pharmaceutical composition as defined herein.

One international unit (IU) of oxytocin is the equivalent of about 1.67 micrograms of pure peptide. Accordingly, a composition of 1 g of oxytocin gel, 400 IU, is equivalent to about 0.67 mg/g (Ph Eur). This is also sometimes referred to as IE.

As an example, a topical composition for use as described herein may comprise about: 1 mg/g oxytocin, about 2 wt % hydroxypropylmethylcellulose in about 25 mM citrate or lactate buffer, and optionally about 1 mg/g benzoic acid.

As an example, a topical composition for use as described herein may comprise about 0.9 mg/g oxytocin, about 1.1 mg/g benzoic acid, and about 2 wt % hydroxypropylmethylcellulose (HPMC) in about 25 mM lactate buffer.

As an example, oxytocin, and/or said one or more fragment(s) and/or variant(s) thereof may be present in an amount of about 0.1 to 1.5 mg/g, such as 0.5 to about 1.5 mg/g, about 0.5 to about 1 mg/g, about 0.5 to about 1.2 mg/g, about 0.2 to about 0.5 mg/g, about 0.1 to about 0.8 mg/g, or about 0.2 to about 1.2 mg/g of the total composition, and said at least one non-ionic cellulose ether may be present in an amount of about 1-3 wt %, such as about 2 wt % or 3 wt % in a pH regulating agent, such as a buffer, said buffer having a concentration of about 25 mM to about 100 mM. As an example, said at least one non-ionic cellulose ether is hydroxypropyl methylcellulose.

As an example, a topical composition for use as described herein may comprise about 1 mg/g oxytocin, about 2 wt % hydroxypropylmethylcellulose (HPMC) in about 25 mM citrate or lactate buffer and optionally about 1 mg/g benzoic acid and wherein the pH of said composition is about 3.

As an example, a topical composition for use as described herein may comprise about 1 mg/g oxytocin, about 2 wt % hydroxypropylmethylcellulose (HPMC) in about 25 mM lactate buffer and wherein the pH of said composition is about 3.8.

As an example, a topical composition for use as described herein may comprise about 0.9 mg/g oxytocin, about 1.1 mg/g benzoic acid, about 2 wt % hydroxypropylmethylcellulose (HPMC) in about 25 mM lactate buffer and wherein the pH of said composition is about pH 3.8.

As an example, a topical composition for use as described herein may comprise hydroxypropylmethylcellulose, a citrate or a lactate buffer, and optionally benzoic acid. In one aspect, said pharmaceutical composition comprises about: 1 mg/g oxytocin, about 2 wt % hydroxypropylmethylcellulose in about 25 mM citrate or lactate buffer, and optionally about 1 mg/g benzoic acid.

As an example, a topical composition for use as described herein may comprise: 1 mg/g oxytocin, 2 wt % hydroxypropylmethylcellulose in a 25 mM lactate buffer, and optionally 1 mg/g benzoic acid.

As an example, a topical composition for use as described herein may comprise: 0.8 mg/g oxytocin, 3 wt % hydroxypropylmethylcellulose in a 22 mM lactate buffer, and optionally 1 mg/g benzoic acid.

As an example, a topical composition for use as described herein may comprise 0.4 mg/g oxytocin, 3 wt % hydroxypropylmethylcellulose in a 22 mM lactate buffer, and optionally 1 mg/g benzoic acid.

As an example, a topical composition for use as described herein may comprise 0.2 mg/g oxytocin, 3 wt % hydroxypropylmethylcellulose in a 22 mM lactate buffer, and optionally 1 mg/g benzoic acid.

A topical composition for use as defined herein, may be hydroxypropylmethylcellulose (HPMC), also referred to as hypromellose. It is also related to herein, a topical composition for use in the prevention and/or treatment of a deficiency in endogenous oxytocin production, wherein said topical composition comprises oxytocin (SEQ ID NO:1) and hydroxypropylmethylcellulose (HPMC). Said topical composition may be for vaginal use.

When a non-ionic cellulose ether is used herein, the concentration of a non-ionic cellulose ether, such as hydroxypropylmethylcellulose (HPMC) is provided in relation to a pH regulating agent of a topical composition, such as a buffer, as presented herein, e.g. 2 wt % or 3 wt % non-ionic cellulose ether in 25 mM buffer provides a certain viscosity due to the amount of the at least one non-ionic cellulose ether present in said buffer. Accordingly, the concentration of the non-ionic cellulose ether is presented in weight % (wt %) in said buffer, or other pH regulating agent. In the total composition, the amount of non-ionic cellulose ether is somewhat less due to the addition of the therapeutic substance (oxytocin and/or one or more fragment(s) and/or variant(s) thereof as disclosed herein) to said composition.

The concentration of oxytocin and/or one or more fragment(s) and/or variant(s) thereof as defined herein is provided in relation to the total composition, e.g. for example in a buffer and a gel such as a non-ionic cellulose ether, if applicable, and optionally other additives such as a preservative. Hence, 1 mg/g oxytocin means that 1 mg oxytocin is present in 1 g of total composition. In addition, 1 mg/ml means that 1 mg of oxytocin is present in 1 ml of total topical composition.

A further object of the present disclosure relates to a method for treating and/or preventing an imbalance in the endogenous production and/or function of oxytocin in a subject, and optionally vaginal atrophy, comprising administrating a therapeutically effective amount of a topical composition comprising oxytocin as defined herein to said subject in need thereof. Said topical composition may be administered to the peripheral blood stream of said subject in a final dosage of between about 15 μg and about 70 μg, such as between about 15 and 30 μg. Such a composition may comprise between about 50 and 400 IU of oxytocin, such as between about 100 and 200 IU. In a method as disclosed herein, said disorder may be selected from the group consisting of oxytocin deficiency, low social ability, anxiety, low energy level, stress sensitivity, mental climacteric disorders, obsessive-compulsive disorder (OCD), borderline, attachment disorders, depression autism and other forms of mental fragility or illness, pain, inflammation, an abusive disorder and/or disturbances of cardiovascular and gastrointestinal function of central origin. Said subject may be a mammal, such as a human being.

The topical compositions as provided herein may in certain circumstances contain substances that extend or strengthen the effects of oxytocin. Such substances could increase the release of oxytocin and/or the member or affinity of oxytocin receptors, such as oestrogen, or drugs having an [alpha]₂-agonistic effect, such as clonidine.

The topical compositions as defined herein are may be encompassed for topical use, such as for vaginal use. Herein, the present disclosure relates to a topical composition comprising oxytocin (SEQ ID NO:1) and hydroxypropylmethylcellulose (HPMC), optionally in combination with buffers, other pH regulating agents or additional components as exemplified herein, such as benzoic acid for vaginal use, for use in the treatment and/or prevention of a dysfunction in the endogenous release of oxytocin.

In all aspects herein, it is to be understood that whenever it is referred to a use in a medical condition, this also refers to the use of oxytocin and/or one or more fragment(s) and/or variant(s) thereof, as defined herein, in the manufacture of a medicament in the form of a topical composition for the treatment and/or prevention of the certain disease, such as exemplified herein, e.g. use of oxytocin and/or one or more fragment(s) and/or variant(s) thereof, in the manufacture of a topical composition for use in treating and/or preventing an imbalance in the endogenous oxytocin production and/or function.

There are different processes described for the synthetic production of oxytocin; commercial processes are for instance described in U.S. Pat. Nos. 2,938,991 and 3,076,797.

In some aspects, the compositions as defined herein can optionally comprise other components, such as inert components or components having a physical or biological function. In some aspects, an antibiotic or an analgesic is additionally present in a composition as provided herein.

The experimental section is only illustrative of the present disclosure and is not intended to limit the scope of the disclosure in any manner.

### EXPERIMENTAL SECTION

### Example 1

### Effects of Intravaginal Administration of Oxytocin

Experimental Set-Up

The study comprised of 64 patients who received daily intravaginal treatment with an oxytocin containing gel or a placebo gel. 24 patients received 400 IU oxytocin, 24 patients received 100 IU oxytocin and 16 received placebo gel. The vagitocin gel comprises of 1 mg of oxytocin per ml hypromellose (HPMC). Benzoic acid is used as preservative and lactic acid to adjust the pH to between about 3 and 4.

The patients were observed after 2 and 7 weeks of treatment.

Results and Conclusions

Mucosal Growth

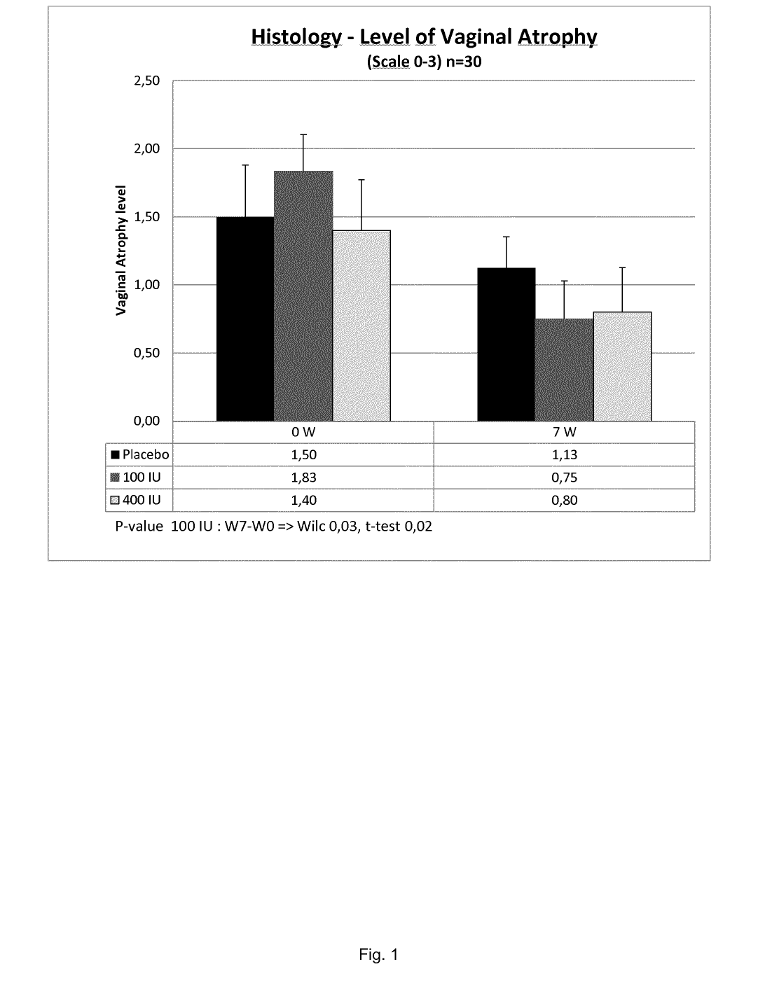

According to histological investigation the mucosa of patients treated with oxytocin was more mature and thicker after 7 weeks than in patients belonging to the placebo group suggesting that oxytocin treatment had stimulated the growth of vaginal mucosa (FIG. 1).

Most Bothersome Symptoms

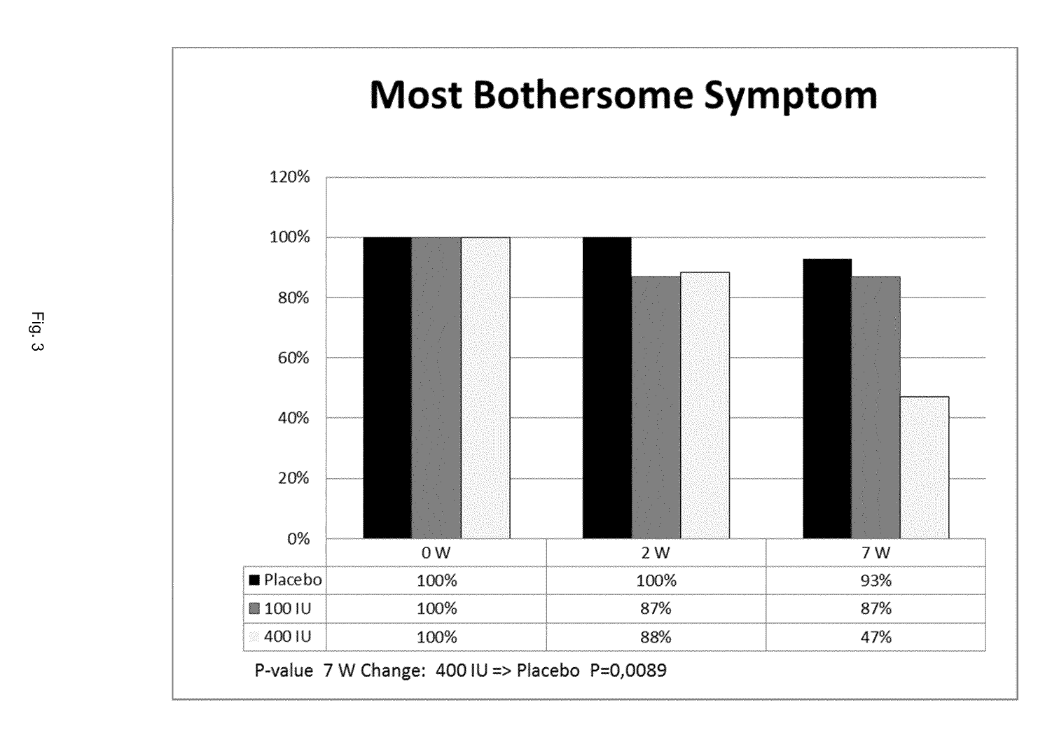

The women participating in the study were asked to grade their symptoms of vaginal atrophy, such as dyspareunia, dysuria, vaginal dryness and itching and irritation at inclusion of the study, and 2 and 7 weeks after initiation of treatment. In addition they were asked to name which of these symptoms bothered them the most (the most bothersome symptom), when they were included in the study. At the following visits, i.e. at 2 and 7 weeks, they were asked if they still experienced “the most bothersome symptom” yes or no. After 7 weeks the women who had received 400 IU of oxytocin intravaginally everyday significantly more often (p=0.008) reported that they did not experience the most bothersome symptom at all than the women having received placebo (FIG. 3).

Blood Plasma Levels of Oxytocin

Blood samples were collected from 20 individuals before and repeatedly after administration of oxytocin during a 90 minute period. Plasma levels of oxytocin rose dose dependently following administration of oxytocin 100 and 400 IU. (FIG. 2). The finding of slightly elevated peripheral oxytocin levels after disappearance of the intravaginally administered oxytocin, suggests that also the secretion of endogenous oxytocin to the circulation was reinforced by the intravaginal treatment.

Swedish Scales of Personality

All participating women filled in a personality inventory, the Swedish Scales of Personality and Women Health Questionnaire (WHQ) before initiation of treatment and after 2 and 7 weeks of treatment with oxytocin or placebo. The Swedish Scales of Personality and the WHQ are both validated inventories.

Summary of Swedish Universities Scales of Personality

Summary of statistically significant results obtained in women receiving 100 or 400 IU of oxytocin versus placebo treated controls.

More Polite and Helpful to Other People

I am always polite, even to unpleasant people
7 v, 400 IU p=0.0938
I always help out when somebody needs me
7 v, 100 IU p=0.0466, 400 IU p=0.2624

More Secure in the Presence of Other People

I often feel uneasy when I meet people I don't know too well
2 v, 100 IU p=0.00951, 400 IU.0734
7 v, 440 IU p=0.0587
I feel best when I keep people at certain distance
7 v, 400 IU p=0.0706

More Self-Secure

I seldom dare to express myself in a discussion because I have the feeling that people
think my views are not worth anything
2 v, 400 IU p=0.0664
7 v, 100 IU p=0.0461, 400 IU p=0.0287
I don't have much self confidence
7 v, 400 IU p=0.0949
I am probably the kind of person who is excessively sensitive and easily hurt
2 v, 100 IU p=0.0781, 400 IU p=0.0958
7 v, 100 IU p=0.1968, 400 IU p=0.1412

More Energy

In order to get something done I have to spend more energy than most others do
2 v, 100 IU p=0.0078, 400 IU p=0.1422
7 v, 100 IU p=0094, 400 IU p=0.0600

Increased Stress Tolerance

I easily feel pressure when told to speed up my work
2 v, 100 IU p=0.0119, 400 IU p=0.0737
4 v, 100 IU p=0.0283
I don't mind being interrupted when I am working with something
2 v, 100 IU p=0.0092, 400 IU p=0.0639

More Calm and Contentment

I often feel restless as if I wanted something without knowing what
2 v, 400 IU p=0.1311
7 v, 400 IU p=0.04

More Heart Beats

Sometimes my heart pounds hard or irregularly for no apparent reason
2 v, 100 IU p=0.0470
7 v, 100 IU p=0.0243, 400 IU p=0.0434

Hence, women receiving oxytocin experienced more wellbeing and less anxiety and felt more self-secure and energetic than those who received placebo gel, indicating that the intravaginal oxytocin treatment had induced a release of endogenous oxytocin in the brain. As the elevation of plasma levels of oxytocin observed in the present study is not sufficient to allow a passage of significant amount of oxytocin through the blood brain barrier, the effect induced is likely to have been indirect via activation of sensory nerves, which in turn leads to a release of oxytocin in the brain.

Karolinska Scales of personality or Swedish Scales of personality (SSP) is a validated inventory which has been used to demonstrate oxytocin linked changes in personality during e.g. breastfeeding. Oxytocin levels observed in response to breastfeeding correlates strongly with lower levels of anxiety and increased levels of social skills as determined by KSP. Uvnäs-Moberg K et al., Acta Obstet Gynecol Scand 69, 301-6, Uvnäs-Moberg K et al., J Psychosom Obstet Gynaecol 1990, 11 261-273, Nissen E, J Psychosom Obstet Gyneaecol 1998, 19, 49-58).

Pharmacokinetics

The amount of oxytocin which is absorbed following intravaginal administration of oxytocin expressed as area under the curve (AUC) can be calculated using the trapezoid method according to standardized pharmacokinetic procedures as the clearance rat for oxytocin (CL) is known (70 L/H).

As oxytocin levels were only measured during a 90 minute period it had to be assumed that the concentration of oxytocin after 2 hours continues to fall according to the half-life of oxytocin.

In this way it could be calculated that 100 and 400 IU of oxytocin gave rise to an AUC corresponding to approximately 156 and 1103 μg/ml/h respectively and that 0.064 and 0.11 of the given dose had been absorbed.

### Example 2

### Initial Skin Penetration Experiments on Oxytocin

Experimental Set-Up

Skin penetration was performed in Franz diffusion cells using dermatomed pig's ear skin (thickness approx. 500 μm and surface area 0.64 cm²). The receptor volume of each cell was 6 mL and citrate buffer, pH 4.5 was used as receptor media. The temperature was maintained at 32° C. and the stirring speed in the receptor chamber was kept at 300 rpm. Sampling was done in triplicate at 24 hours intervals for 4 days, i.e. 24, 48, 72 and 96 h. 1.5 mL of receptor media was taken and replaced with the same volume of citrate buffer. Immediately after collection the samples were kept at −20° C.

A water solution of oxytocin was prepared in a concentration of 4000 IU/g, which corresponds to ca 6.8 mg/g. The solution was pH adjusted with a 50 mM citrate buffer to 4.0 in order to achieve reasonable chemical stability. The oxytocin solutions were added in excess, ca 1 g, on the donor side of the skin and covered to prevent evaporation.

The samples were thawed just prior analysis by HPLC, method M272-0. The limit of quantification (QL) is 12.5 ng/mL or 1.25 ng injected amount.

Results and Discussion

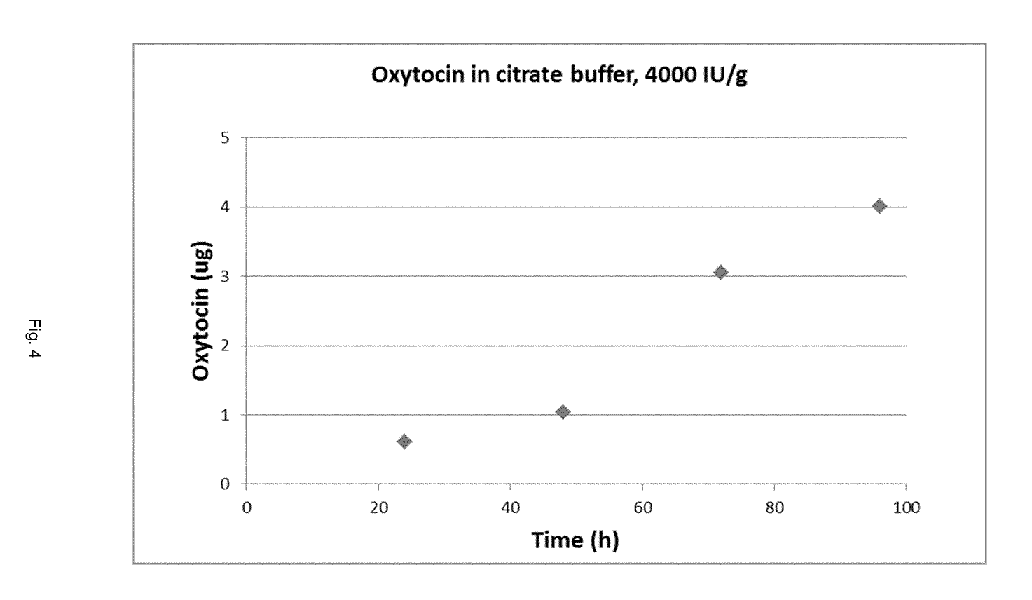

A typical lag phase followed by an increase in concentration over time was found and displayed in FIG. 4. After 96 hours 4 μg oxytocin was detected in the receptor chamber. The steady state flux was determined to 0.1 μg/h·cm²=100 ng/hour/cm².

This gives, for a 4000 IU/g formulation a total of 200 IU oxytocin. 200 IU equals approx. 0.4 mg oxytocin, and this indicates that after 24 h (0.6/400 μg) 0.1% has penetrated the skin. After 96 h using the same calculation (4/400 μg) approximate 1% has penetrated the skin.

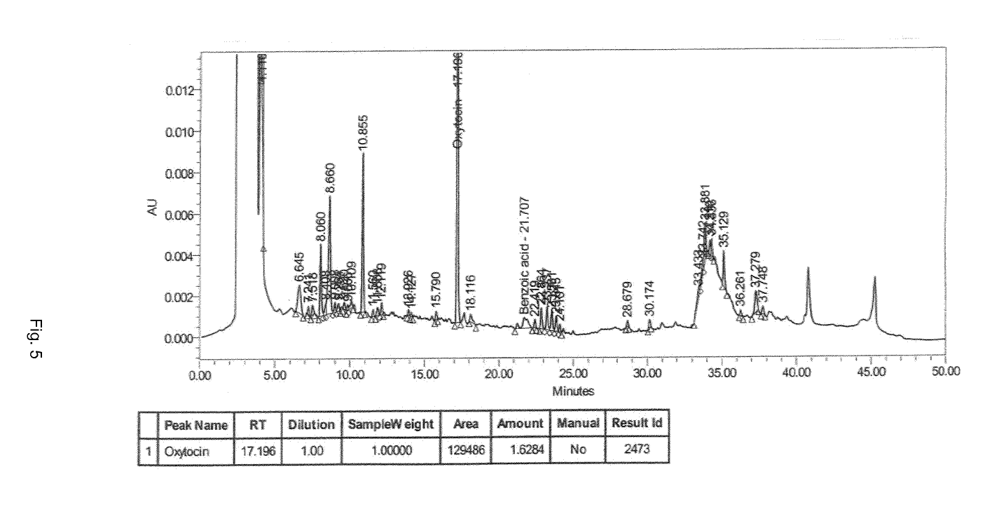

These data on a simple water solution encouraged for further evaluation of developed topical oxytocin formulations. The analytical method showed sufficient QL and separation for the determination of oxytocin when analyzing the water solution containing 4000 IU/g for the time interval used. A chromatogram for the water solution after 48 h is presented in FIG. 5. It is therefore demonstrated that it is possible to measure the amount of oxytocin in a solution by the above described method.

### Example 3

### Calculation of the Bioavailability of Vaginally Administered Oxytocin

Introduction

The knowledge of the pharmacokinetic (PK) characteristics of oxytocin is still rather limited. In 1995, De Groot and colleagues studied the PK of oxytocin in six male subjects following an intravenous dose of 1 IU (1.7 μg) (De Groot A N et al., 1995). The oxytocin PK was described by a two-compartment model with a reported total body CL of 67.1 L/h, a volume of distribution (V) of 33.2 L, and a terminal elimination half-life (t_{1/2}) of 0.33 h. This is in accordance to others, reporting the CL to be in the rage of 70-90 L/h as assessed in healthy male subjects (Amico J A et al., 1987, Dawood M Y et al, 1980).

Objective

The objective of the present analysis was to estimate the bioavailability of oxytocin following vaginal administration of 100 (170 μg) or 400 IU (680 μg) oxytocin vaginal gel using data from study OXYPEP002 (Clinical trial protocol OXYPEP002). The oxytocin vaginal gel is Vagitocin® gel.

Vagitocin® is a clear gel in the strength 400 IU/g. Vagitocin gel is provided in single dose glass syringes containing 1 g. The pH of the gel is adjusted to 3.75 and benzoic acid is used as preservative.

TABLE 1
Composition of 1 g of Vagitocin gel, 400
IU, equivalent to 0.67 mg/g (Ph Eur)

| | Quantity | | | Reference to |
|---|---|---|---|---|
| Ingredients | mg | % w/w | Function | Standard |
| Oxytocin | 0.769* | 0.0769* | Drug substance | Ph Eur |
| Benzoic acid | 1 | 0.1 | Preservative | Ph Eur |
| Lactic acid | 0.0022 | 0.00022 | Buffering agent | Ph Eur |
| Sodium hydroxide | q.s.** | q.s.** | Buffering agent | Ph Eur |
| 5M | | | | |
| Hypromellose | 30 | 3.0 | Viscosity agent | Ph Eur |
| (Benecel) | | | | |
| Purified water | q.s. *** | q.s. *** | Solvent | Ph Eur |

*Based on the potency of the oxytocin batch used (batch no. 120511, Grindex), which was 520 IU/mg (400/520 = 0.769)
**To a pH of 3.75
*** To a final weight of 1000 mg

Data

Data from study OXYPEP002 was used for the analysis. Study OXYPEP002 was conducted with the primary objective to investigate the dose-relationship of topical Vagitocin on the vaginal mucosal membrane (Clinical trial protocol OXYPEP002). OXYPEP002 was a double-blind, randomized, placebo controlled trial, with a parallel group design. Vagitocin (oxytocin gel) in two different doses (100 IU and 400 IU) was administered topically once daily during 7 weeks. A placebo gel with the same composition was used as comparator. In total 64 patients were to be included in the study. At the baseline visit (visit 1), blood sampling for oxytocin analysis were drawn for the first 24 included subjects. This sample represents the true baseline oxytocin observation. For the same 24 subjects, blood sampling for oxytocin analysis were also drawn at visit 2 (2 weeks ±2 days) with sampling pre-dose and at 30 min, 60 min and 120 min post-dose. Only subjects that completed the full sampling schedule were included in the analysis. The calculations were based on average blood oxytocin measurements for each dose level and sampling time point (Table 2).

TABLE 2
Average blood oxytocin levels (pg/ml)
as reported in study OXYPEP002.

| Dose | No of | Baseline | Pre-dose | 30 min | 60 min | 120 min |
|---|---|---|---|---|---|---|
| level | subjects | Visit 1 | Visit 2 | Visit 2 | Visit 2 | Visit 2 |
| Placebo | 5 | 20.4 | 18.6 | 22.6 | 15.6 | 17.2 |
| 100 IU | 10 | 17.9 | 37.0 | 133 | 120 | 99.0 |
| 400 IU | 9 | 15.1 | 60.6 | 816 | 630 | 378 |

Bioavailability Calculations

a. Systemic Oxytocin Exposure Following Vaginally Administered Oxytocin

The systemic oxytocin exposure following vaginally administered oxytocin was assessed by calculating the area under the blood concentration versus time curve (AUC) according to the trapezoidal rule. The data from visit 2 were used where the pre-dose sample was assumed to represent a new baseline observation.

The blood concentration of oxytocin had not returned to the baseline observation at the lasts sampling time point (120 min post-dose). Two approaches to calculate the oxytocin exposure after the 120 min post-dose sampling (e.g. the residual area) were used i) assuming the oxytocin concentration to continue to decline at the same rate after 120 min as between the 60 min and 120 min post-dose samples or ii) assuming the oxytocin concentration after 120 min to decline according to the t_{1/2} reported in the literature following iv oxytocin administration.

The endogenous oxytocin level (AUC_{baseline}) was calculated and subtracted from the total AUC to achieve the oxytocin exposure following the vaginally administered dose (AUC_{vaginal}).

Calculated AUCs for the two dose levels using the two approaches to calculate the residual area are presented in Table 3.

TABLE 3
Calculated AUC (pg/ml · h) for vaginally administered oxytocin at two dose levels.

| Dose | | | | | | |
|---|---|---|---|---|---|---|
| level | AUC_{0-30 min} | AUC_{30-60 min} | AUC_{60-120 min} | AUC_{residual} | AUC_{baseline} | AUC_{vaginal} |
| 100 IU | 42.6 | 63.3 | 110 | 346¹ | 263¹ | 299¹ |
| | | | | 32.2² | 91.5² | 156² |
| 400 IU | 219 | 362 | 504 | 783¹ | 338¹ | 1530¹ |
| | | | | 193² | 174² | 1103² |

¹residual area calculated according to alternative i
²residual area calculated according to alternative ii

b. Oxytocin Bioavailability

The bioavailability was calculated according to equation 1. As no intravenous dose was administered in study OXYPEP002, AUCᵢᵥ was calculated according to available information in the literature (De Groot A N et al. 1995, Amico J A et al. 1987, Dawood M Y et al. 1980) according to equation 2. An assumed intravenous dose of 1 μg and a CL of 70 L/h would result in an AUCᵢᵥ of 14.3 μg/ml·h. According to this the bioavailability was calculated to be in the range of 0.064-0.16 (Table 4).

$$\begin{matrix}F = \frac{{AUC}_{vaginal}}{{AUC}_{iv}} \times \frac{{Dose}_{iv}}{{Dose}_{vaginal}} & Equation 1 \\ {AUC}_{iv} = \frac{{Dose}_{iv}}{CL} & Equation 2\end{matrix}$$

TABLE 4
Calculated bioavailability following vaginally
administered oxytocin at two dose levels.

| Dose level | Bioavailability |
|---|---|
| 100 IU | 0.12¹ |
| | 0.064² |
| 400 IU | 0.16¹ |
| | 0.11² |

¹residual area calculated according to alternative i
²residual area calculated according to alternative ii

The bioavailability following vaginally administered oxytocin was in this study estimated to be in the range of 0.064-0.16. This is somewhat higher than expected.

The calculation of the bioavailability was not one of the objectives of study OXYPEP002 and due to limitations in the study design the results here should be regarded as a rough estimate rather than the true value. The blood sampling in OXYPEP002 was sparse and limited to a 2 h sampling period. Since the oxytocin concentration at the latest sample was higher than the baseline observation assumptions were needed regarding the residual oxytocin exposure. Further, when comparing the decline in oxytocin concentration between the last two samples to the reported elimination rate of oxytocin it was indicated that the absorption process might not have been completed at the latest sampling time point. Two alternative calculations were thus made. In the first alternative, the oxytocin level were assumed to continue to decline with a rate according the decline in concentration between the last two observations, thus assuming the absorption process to continue at the same extent until the baseline observation was reached. This would probably lead to an overestimation of the drug exposure and the bioavailability for the vaginal route of administration. The other alternative used was to assume that the absorption process was completed at the latest sampling time point and that the drug concentration would thereafter decline according the half-life of oxytocin previously reported in the literature. If the absorption process was not completed this would lead to an underestimation of the oxytocin exposure and bioavailability following the vaginal exposure. The true value probably lies somewhere between these two extreme situations. Another uncertainty lies in the fact that no intravenous dose was administered in this study. The information regarding oxytocin exposure following intravenous administration was thus extracted from the literature and differences in analysis methods between studies might influence the results.

### Example 4

### Example of how Skin Penetration May be Calculated: Passive Diffusion of Drugs Across the Human Skin

Since stratum corneum is dead, there exists no active transport across this region of the skin. Transdermal drug permeation is, thus, dependent on passive diffusions. The passive diffusion of a drug molecule across SC obeys Fick's first law, i.e. the diffusion rate per unit area is proportional to the concentration gradient:

$$J = - D \frac{\partial c}{\partial x}$$

where J is the diffusion rate per unit of surface area, D is the diffusion coefficient (dependent on the solute and the surrounding conditions, such as membrane type and temperature), C is the concentration and x is the space coordinate.

In diffusion testing with Franz cells, a membrane separates a donor and a receptor compartment. If the concentration in the donor compartment is in large excess one can suspect unidirectional diffusion. The drug permeation is then described by the following equation:

$$J = \frac{D {C}_{0} P}{h}$$

where J is the steady state flux, D is the diffusion coefficient, C₀ is the drug concentration (assumed to be constant), P is the partition coefficient between the membrane and the drug vehicle and h is the thickness of the membrane. Describing diffusion in vivo is more complex since each layer of the skin contributes to the diffusional resistance. However, if one layer has a much greater resistance than the others then the diffusion can be approximately explained by the equation given for diffusion testing. The parameters to use are then those for the membrane with greatest resistance.

### REFERENCES

Uvnäs-Moberg K, Widström A M, Werner S, Matthiesen A S, and Winberg J
Oxytocin and prolactin levels in breast-feeding women. Correlation with milk yield and duration of breastfeeding
Acta Obstet Gynecol Scand 69, 301-6
Uvnäs-Moberg K, Widström A M, Nissen E and Björvell H.
Personality traits in women 4 days postpartum and their correlation with plasma levels of oxytocin and prolactin
J Psychosom Obstet Gynaecol 1990, 11 261-273
Nissen E, Gustaysson P, Widström A M and Uvnäs Moberg K
Oxytocin, prolactin, milk production and their relationship with personality traits in women after vaginal delivery or Cesarean section.
J Psychosom Obstet Gyneaecol 1998, 19, 49-58
De Groot A N, Vree T B, Hekster Y A, Pesman G J, Sweep F C, Van Dongen P J, Van Roosmalen J. Bioavailability and pharmacokinetics of sublingual oxytocin in male volunteers. J Pharm Pharmacol. 1995 July; 47(7):571-5.
Amico J A, Ulbrecht J S, Robinson A G. Clearance studies of oxytocin in humans using radioimmunoassay measurements of the hormone in plasma and urine. J Clin Endocrinol Metab. 1987 February; 64(2):340-5.
Dawood M Y, Ylikorkala O, Trivedi D, Gupta R. Oxytocin levels and disappearance rate and plasma follicle-stimulating hormone and luteinizing hormone after oxytocin infusion in men. J Clin Endocrinol Metab. 1980 February; 50(2):397-400.
Clinical trial protocol OXYPEP002. A double-blind, placebo controlled single centre trial to evaluate the dose-relationship of the effects of vaginally administered oxytocin on the vaginal mucosal membrane in postmenopausal women with vaginal atrophy.

1.-33. (canceled)

34. A method for increasing endogenous oxytocin production in a subject comprising topically administering a composition comprising oxytocin and/or one or more fragment(s) and/or variant(s) thereof to said subject in an amount sufficient to increase the production of oxytocin in the subject without inhibiting endogenous oxytocin production; wherein
said one or more fragment(s) and/or variant(s) of oxytocin corresponds to
X1-X2-X3-X4-Asn-Cys X5 X6 X7 X8 N H2 (SEQ ID NO:2)
wherein
X1 is selected from the group consisting of Cys and nothing;
X2 is selected from the group consisting of Tyr, Phe, and nothing;
X3 is selected from the group consisting of Ile, Val, Hoph, Phe, Cha, and nothing;
X4 is selected from the group consisting of Gln, Ser, Thr, Cit, Arg, and Daba;
X5 is selected from the group consisting of Pro and nothing;
X6 is selected from the group consisting of Ile, Leu, nothing, Val, Hos, Daba, Thr, Arg, and Cit;
X7 is selected from the group consisting of Gly, nothing, and Ala;
X8 is selected from the group consisting of Gly and nothing; and
SEQ ID NO: 2 is not SEQ ID NO: 7.

35. A method according to claim 34, wherein said subject suffers from a disorder selected from the group consisting of: oxytocin deficiency, low social ability, anxiety, low energy level, stress sensitivity, mental climacteric symptoms, obsessive-compulsive disorder (OCD), borderline, attachment disorders, depression, autism and other forms of mental fragility or illness, pain, inflammation, an abusive disorder and/or disturbances of cardiovascular and gastrointestinal function of central origin.

36. A method according to claim 34, wherein said disorder is an oxytocin deficiency.

37. A method according to claim 34, wherein said composition is administered in a manner that achieves a dosage of said oxytocin and/or one or more fragment(s) and/or variant(s) thereof entering the blood of about 15-70 μg.

38. A method according to claim 34, wherein said composition is administered in a manner that achieves a dosage of said oxytocin and/or one or more fragment(s) and/or variant(s) thereof entering the blood of about 15-30 μg.

39. A method according to claim 34, wherein said composition is administered in a manner that achieves a dosage of said oxytocin and/or one or more fragment(s) and/or variant(s) thereof entering the blood of about 20-30 μg.

40. A method according to claim 34, wherein said composition is administered in a manner that achieves a dosage of said oxytocin and/or one or more fragment(s) and/or variant(s) thereof of about 15-40 μg.

41. A method according to claim 34, wherein said composition is administered in a manner that achieves a dosage of said oxytocin and/or one or more fragment(s) and/or variant(s) thereof of about 20 μg.

42.-43. (canceled)

44. A method according to claim 34, wherein said topical administration comprises epicutaneous, sublingual, buccal, vaginal, or anal administration.

45.-49. (canceled)

50. A method according to claim 34 wherein the subject suffers from vaginal atrophy.

51. A method according to claim 34, wherein said composition is administered topically once daily.

52. (canceled)

53. A method according to claim 34, wherein said one or more fragment(s) and/or variant(s) of oxytocin is/are selected from the group consisting of the peptides corresponding to SEQ ID NO:3-24.

54. A method according to claim 34, wherein said composition has a pH of from about 3 to about 5.

55. A method according to claim 34, wherein said composition further comprises an epithelium penetration enhancer.

56. A method according to claim 55, wherein said vaginal epithelium penetration enhancer is selected from the group consisting of propylene glycol (PG), SDS, isopropyl myristate, ethanol, diethylene glycol ethyl ether and 1 cineole.

57. A method according to claim 34, wherein said composition further comprises at least one non-ionic cellulose ether.

58. A method according to claim 57, wherein said non-ionic cellulose ether is selected from the group consisting of methyl cellulose (MC), hydroxypropylmethylcellulose (HPMC), hydroxypropylcellulose (HPC), hydroxypropylmethylcellulose (HPMC), hydroxyethylethylcellulose (HEEC) and hydroxyethylmethyl cellulose (HEMC).

59. A method according to claim 58, wherein said non-ionic cellulose ether is hydroxypropylmethylcellulose (HPMC).

60. A method according to claim 34, wherein said composition comprises oxytocin (SEQ ID NO:1) and hydroxypropylmethylcellulose (HPMC).

61. A method according to claim 34, wherein said composition comprises:
i) about 0.1 to 2 mg/ml oxytocin and/or one or more fragment(s) and/or variant(s) of oxytocin;
ii) a non-ionic cellulose ether;
said composition having a pH of about 3-5.

62. A method according to claim 61, wherein said composition comprises about 0.17-1 mg/ml of oxytocin.

63. A method according to claim 34, wherein said composition comprises:
i) about 1 to 15 mg/ml oxytocin; and
ii) a non-ionic cellulose ether;
said composition having a pH of about 3-5.

64. A method according to claim 63, wherein said composition comprises about 1.7-10 mg/ml of oxytocin.

65. A method according to claim 61, wherein said non-ionic cellulose ether is hydroxypropylmethyl cellulose.

66. A method according to claim 61, wherein said composition further comprises an epithelium penetration enhancer, such as propylene glycol (PG), SDS, isopropyl myristate, ethanol, diethylene glycol ethyl ether and/or cineole.

67.-100. (canceled)
